(19) United States
(12) Patent Application Publication
(10) Pub. No.: US 2014/0038991 A1
(43) Pub. Date: Feb. 6, 2014

(54) Protein Kinase Inhibitors

(71) Applicants: Chunrong Yu, Glen Allen, VA (US); Yong Huang, Shenzhen (CN)

(72) Inventors: Chunrong Yu, Glen Allen, VA (US); Yong Huang, Shenzhen (CN)

(73) Assignee: Astar Biotech LLC, Richmond, VA (US)

(21) Appl. No.: 13/567,144

(22) Filed: Aug. 6, 2012

(51) Int. Cl. C07D 471/04; A61K 45/06; A61K 31/519

(52) U.S. Cl. CPC C07D 471/04; A61K 31/519; A61K 45/06
USPC 514/264.11; 544/279

(57) ABSTRACT

The present invention is directed to compounds of the Formula I

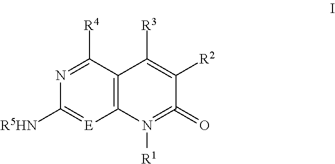

as well as pharmaceutically acceptable salts, hydrates, isomers, or solvates thereof, wherein the variables are described herein. The present invention further relates to pharmaceutical compositions which comprise the compounds of Formula I, and to methods for inhibiting protein kinase and methods of treating diseases, such as cancers, inflammation.

### FIELD OF THE INVENTION

The present invention describes bicyclic heterocycles with inhibitory activity on protein kinases and a pharmaceutical composition for preventing or treating diseases involving abnormal cell growth.

### BACKGROUND OF THE INVENTION

The phosphatidylinositol 3-kinase (PI3K)/mammalian target of rapamycin (mTOR) pathway is one of the most commonly activated cell signaling pathways in human cancers. This pathway is known to play a key role in numerous cellular functions including proliferation, adhesion, migration, invasion, metabolism, and survival.

PI3Ks catalyse the phosphorylation of PtdIns(4,5)P2 (PIP2) to PtdIns(3,4,5)P3 (PIP3). PIP3 propagates intracellular signaling by directly binding pleckstrin homology (PH) domain-containing serine/threonine kinase Akt into close proximity. PIK3CA encodes the p110α catalytic subunit of class I PI3K and its alterations have been observed in several solid tumor malignancies. Most of these mutations cluster to two hot spot regions in exons 9 and 20. Exon 9 encodes the helical domain of p110α, and the mutations may disrupt the regulation of kinase activity by p85 and increase the catalytic activity (Shaw R, Cantley L. Ras, PI(3)K and mTOR signalling controls tumour cell growth. Nature. 2006; 441:424-30. Vivanco I, Sawyers C. The phosphatidylinositol 3-kinase-Akt pathway in human cancer. Nature Reviews Cancer. 2002; 2:489-501. Courtney K, Corcoran R, Engelman J. The PI3K pathway as drug target in human cancer. Journal of Clinical Oncology. 2010; 28:1075-83).

When PI3K functions normally, it regulates key cell functions such as growth, motility, proliferation, differentiation, and survival. But, when the pathway is dysregulated such as mutations in PI3K or other genes such as PTEN in the pathway, it can contribute to the development of many different types of cancers, such as breast, ovarian, endometrial, lung, colon, etc. Inhibition of key signaling proteins in the pathway therefore represents a valuable targeting strategy for diverse cancers. Current research is focused on how to control cancer cell growth by inhibiting abnormal PI3K signaling (Kong D, Yamori T. Phosphatidylinositol 3-kinase inhibitors: promising drug candidates for cancer therapy. Cancer science. 2008; 99:1734-40). In this regard, several potent and selective PI3K/mTOR inhibitors, including PF-04691502, BEZ-235, GDC-0980, and PKI-587 have recently entered clinical trials (Ben Markman, Rodrigo Dienstmann, Josep Tabernero. Targeting the PI3K/Akt/mTOR Pathway—Beyond Rapalogs. Oncotarget, 2010, 1, 7: 530-543).

The mTOR consists of two distinct complexes, mTORC1 and mTORC2, which differ in their distinct subunit compositions, substrates, and activation mechanisms. mTORC1, the sensitive target of rapamycin, phosphorylates downstream targets of S6K1 (p70S6K1) and 4E-BP1 which control the cap-dependent protein translation. mTORC2 is insensitive to rapamycin, and its main substrates are AKT and related kinases (Fingar D, Blenis J. Target of rapamycin (TOR): an integrator of nutrient and growth factor signals and coordiantor of cell growth and cell cycle progression. Oncogene 2004; 23:3151-3171. Proud C. mTORC1 signalling and mRNA translation. Biochem Soc Trans 2009; 37:227-231. Ruggero D, Montanaro L, Ma L, Xu W, et al. The translation factor eIF-4E promotes tumor formation and cooperates with c-Myc in lymphomagenesis. Nat Med 2004; 10:484-486. Sarbassov D, Guertin D, Ali S, Sabatini D. Phosporylation and regulation of Akt/PKB by the Rictor-mTOR complex. Science 2005; 307:1098-1101).

mTOR has been reported to play a crucial role in cancer cells by promoting cell growth and survival. Recently, new selective ATP-competitive mTOR kinase inhibitors (mTORKis) have been developed that are able to completely suppress both mTORC1/C2 complex-mediated signaling, thereby suppressing the feedback activation of AKT (Wan X, Harkavy B, Shen N, Grohar P and Helman L. Rapamycin induces feedback activation of Akt signaling through an IGF-1R-dependent mechanism. Oncogene 2007; 26:1932-1940. Choo A, Yoon S, kim S, et al. Rapamycin differentially inhibits S6Ks and 4E-BP1 to mediate cell type-specific repression of mRNA translation. Proc Natl Acad Sci USA 2008; 105:17414-17419. Phung T, Ziv K, Dabydeen D, Eyiah-Mensah G, et al. Pathological angiogenesis is induced by sustained Akt signaling and inhibited by rapamycin. Cancer Cell 2006; 10:159-170). Novel mTORC1/C2 inhibitors such as OSI027, AZD8055 and INK-128 have been tested in clinic for advanced tumors.

### SUMMARY OF THE INVENTION

The present invention relates to compounds which have protein kinase inhibitory activity, which are valuable pharmaceutically active compounds for the therapy to treat abnormal cell growth diseases, for example tumors in cancer patients.

In one aspect, the present invention provides compounds of Formula I:

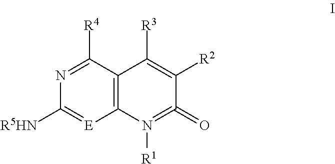

wherein:

R¹ is C₁-C₆ alkyl, C₃-C₇ cycloalkyl, C₂-C₆ heterocyclic alkyl, C₆-C₁₀ aryl, or C₃-C₁₀ heteroaryl, wherein said C₁-C₆ alkyl, C₃-C₇ cycloalkyl, C₂-C₆ heterocyclic alkyl, C₆-C₁₀ aryl, and C₃-C₁₀ heteroaryl are optionally and independently substituted with —OR, C₁-C₆ alkyl, C₃-C₇ cycloalkyl, C₂-C₆ alkynyl, C₆-C₁₀ aryl, C₂-C₆ heterocyclic alkyl, or C₃-C₁₀ heteroaryl, wherein said C₁-C₆ alkyl, C₃-C₇ cycloalkyl, C₂-C₆ alkynyl, C₆-C₁₀ aryl, C₂-C₆ heterocyclic alkyl, and C₃-C₁₀ heteroaryl are further optionally and independently substituted with 0-3 groups selected from halogen, —OR⁶, C₁-C₆ alkyl, C₃-C₇ cycloalkyl, —CN, —COOR⁷, —CONR⁸R⁹, —NR¹⁰C(O)R¹¹, —NR¹²S(O)ₓR¹³, —S(O)ₓNR¹⁴R¹⁵, and —NR¹⁶R¹⁷;

R is H, C₁-C₆ alkyl, C₂-C₆ heterocyclic alkyl, or C₃-C₇ cycloalkyl, wherein said C₁-C₆ alkyl, C₂-C₆ heterocyclic alkyl and C₃-C₇ cycloalkyl are optionally and independently substituted with 0-3 groups selected from halogen, C₂-C₆ heterocyclic alkyl, C₆-C₁₀ aryl, C₃-C₁₀ heteroaryl, —OR⁶, —NR¹⁶R¹⁷, C₁-C₆ alkyl, C₃-C₇ cycloalkyl, —CN, —COOR⁷, —CONR⁸R⁹, —NR¹⁰C(O)R¹¹, —NR¹²S(O)ₓR¹³, —S(O)ₓNR¹⁴R¹⁵, and —NR¹⁶R¹⁷;

R² is C₂-C₆ heterocyclic alkyl, C₆-C₁₀ aryl, —NH—C₆-C₁₀ aryl or C₃-C₁₀ heteroaryl, wherein said C₂-C₆ heterocyclic alkyl, C₆-C₁₀ aryl, —NH—C₆-C₁₀ aryl and C₃-C₁₀ heteroaryl are optionally and independently substituted with 0-3 groups selected from —OR⁶, halogen, —COOR⁷, —CONR⁸R⁹, —NR¹⁰C(O)R¹¹, —NR¹²S(O)ₓR¹³, —S(O)ₓNR¹⁴R¹⁵, —NR¹⁶R¹⁷, —CN, C₂-C₆ alkenyl, C₂-C₆ alkynyl, C₃-C₇ cycloalkyl, and C₁-C₆ alkyl;

R³ and R⁴ are independently H, C₁-C₆ alkyl, C₂-C₆ alkynyl, C₃-C₇ cycloalkyl, C₂-C₆ heterocyclic alkyl, C₆-C₁₀ aryl, halogen, or —CN;

R⁵ is C₁-C₆ alkyl, C₂-C₆ heterocyclic alkyl, or H;

E is CH or N;

each R⁶, R⁷, R⁸, R¹², R¹⁴, and R¹⁶ is independently H, C₁-C₆ alkyl, C₃-C₇ cycloalkyl, C₂-C₆ heterocyclic alkyl, C₆-C₁₀ aryl, or C₃-C₁₀ heteroaryl;

each R⁹, R¹⁰, R¹¹, R¹³, R¹⁵, and R¹⁷ is independently H, C₆-C₁₀ aryl, C₃-C₁₀ heteroaryl, C₂-C₆ heterocyclic alkyl, C₁-C₆ alkyl, or C₃-C₇ cycloalkyl, wherein said aryl, heteroaryl, and heterocyclic alkyl are optionally and independently substituted with 0-3 groups selected from halogen, —OR⁶, C₁-C₆ alkyl, C₃-C₇ cycloalkyl, —CN, and —COOR⁷;

each x is 0, 1 or 2;

wherein said heteroaryl is a 5- to 10-membered mono- or bicyclic ring containing 1-5 heteroatoms selected from O, S and N, said heterocyclic alkyl is a 3- to 10-membered mono- or bicyclic ring containing 1-5 heteroatoms selected from O, S and N, in which the point of attachment may be carbon or nitrogen;

or a pharmaceutically acceptable salt, hydrate, or solvate thereof.

In a further aspect, the present invention provides a pharmaceutical composition comprising at least one compound of Formula I, or a salt, hydrate, isomer, or solvate thereof, and one or more pharmaceutically acceptable carriers and/or additives.

In a further aspect, the present invention provides a method for inhibiting protein kinase comprising administering a therapeutically effective amount of a compound of Formula I, or a salt, hydrate, isomer, or solvate thereof, to a patient in need thereof.

In still a further aspect, the present invention provides a method of treating abnormal cell growth in a human patient in need thereof, comprising administering a therapeutically effective amount of a compound of Formula I, or a salt, hydrate, isomer, or solvate thereof.

In still a further aspect, the present invention provides a method of treating inflammation in a human patient in need thereof, comprising administering a therapeutically effective amount of a compound of Formula I, or a salt, hydrate, isomer, or solvate thereof.

In still a further aspect, the present invention provides a use of a compound of Formula I, or a salt, hydrate, isomer, or solvate thereof, as an active ingredient, for the preparation of a medicament for the treatment of abnormal cell growth or inflammation.

### DETAILED DESCRIPTION OF THE INVENTION

One aspect of the present invention is to provide novel compounds according to Formula I shown and described above. Specifically, the compounds of the invention are protein kinase inhibitors. As a result, this invention provides novel compounds according to Formula I, as well as pharmaceutically acceptable salts, hydrates, isomers or solvates thereof. Values and particular values for the variables in Formula I are provided in the following paragraphs.

In an embodiment, R⁴ is Me; R⁵ is H; E is N; R³ is H; and all other variables in Formula II are as previously defined in Formula I:

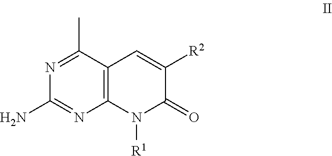

In another embodiment, R² is substituted benzene or pyridine as depicted in Formula III, R¹ is as previously defined in Formula I:

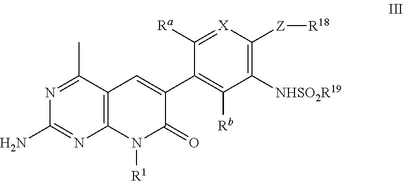

In Formula III,

Z is a bond, O, NH, NR, S, SO, or SO₂;

X is N or CH;

R¹⁸ is C₁-C₆ alkyl, C₃-C₇ cycloalkyl, C₂-C₆ heterocyclic alkyl, or H;

R¹⁹ is C₆-C₁₀ aryl, C₃-C₁₀ heteroaryl, C₁-C₆ alkyl, C₂-C₆ heterocyclic alkyl, or C₃-C₇ cycloalkyl, wherein said C₆-C₁₀ aryl, C₃-C₁₀ heteroaryl, C₁-C₆ alkyl, C₂-C₆ heterocyclic alkyl, and C₃-C₇ cycloalkyl are optionally and independently substituted with 0-3 groups selected from halogen, —OR, C₁-C₆ alkyl, C₂-C₆ heterocyclic alkyl, C₃-C₇ cycloalkyl, —CN, —COOR⁷, —CONR⁸R⁹, —NR¹⁰C(O)R¹¹, —NR¹²S(O)ₓR¹³, —S(O)ₓNR¹⁴R¹⁵, and —NR¹⁶R¹⁷; wherein R, R⁷, R⁸, R⁹, R¹⁰, R¹¹, R¹², R¹³, R¹⁴, R¹⁵, R¹⁶, and R¹⁷ are as previously defined in Formula I.

Each x is 0, 1 or 2;

R^{a} and R^{b} are independently C₁-C₆ alkyl, C₃-C₇ cycloalkyl, C₂-C₆ heterocyclic alkyl, H, halogen, or —CN.

In another embodiment, R^{a}═R^{b}═H, Z═O, X═N, as depicted in Formula IV:

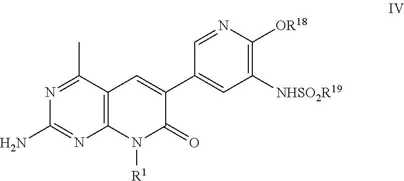

In Formula IV, R¹⁸ and R¹⁹ are as previously defined in Formula III. The definition of R¹ and R is the same as previously defined in Formula I.

In another embodiment, R¹ is C₂-C₆ heterocyclic alkyl as depicted in Formula V, wherein R¹⁸ and R¹⁹ are as previously defined in Formula IV.

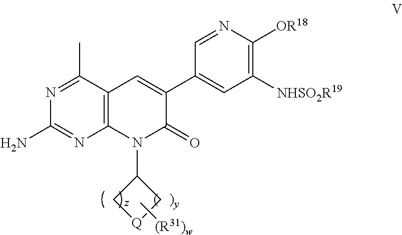

In Formula V:

Q is O or NR³²;

each R³¹ is independently C₁-C₆ alkyl, C₃-C₇ cycloalkyl, C₂-C₆ heterocyclic alkyl, C₆-C₁₀ aryl, C₃-C₁₀ heteroaryl, C₂-C₆ alkynyl, —CN, —OR, —COOR²⁰, —CONR²¹R²², —NR²³C(O)R²⁴, —NR²⁵S(O)ₘR²⁶, —S(O)ₘNR²⁷R²⁸, or —NR²⁹R³⁰, wherein said C₁-C₆ alkyl, C₃-C₇ cycloalkyl, C₂-C₆ heterocyclic alkyl, C₆-C₁₀ aryl, C₃-C₁₀ heteroaryl, and C₂-C₆ alkynyl are further optionally and independently substituted with up to 5 groups selected from halogen, C₁-C₆ alkyl, —CN, —OR, —COOR²⁰, —CONR²¹R²², —NR²³C(O)R²⁴, —NR²⁵S(O)ₘR²⁶, —S(O)ₘNR²⁷R²⁸, and —NR²⁹R³⁰;

y is 1, 2, or 3;

z is 1, 2, or 3;

w is 0, 1, or 2;

R³² is C₁-C₆ alkyl, C₃-C₇ cycloalkyl, C₂-C₆ heterocyclic alkyl, C₆-C₁₀ aryl, C₃-C₁₀ heteroaryl, —COOR²⁰, —CONR²¹R²², or —S(O)ₘNR²⁷R²⁸, wherein said C₁-C₆ alkyl, C₃-C₇ cycloalkyl, C₂-C₆ heterocyclic alkyl, C₆-C₁₀ aryl, and C₃-C₁₀ heteroaryl are further optionally and independently substituted with 1-5 groups selected from halogen, C₁-C₆ alkyl, —CN, —OR, —COOR²⁰, —CONR²¹R²², —NR²³C(O)R²⁴, —NR²⁵S(O)ₘR²⁶, —S(O)ₘNR²⁷R²⁸, and —NR²⁹R³⁰;

Each m is independently 0, 1, or 2.

The definition of R is the same as previously defined in Formula I.

Each R²⁰, R²¹, R²², R²³, R²⁴, R²⁵, R²⁶, R²⁷, R²⁸, R²⁹, R³⁰ is independently H, C₆-C₁₀ aryl, C₃-C₁₀ heteroaryl, C₂-C₆ heterocyclic alkyl, C₁-C₆ alkyl, or C₃-C₇ cycloalkyl, wherein said aryl, heteroaryl, and heterocyclic alkyl are optionally and independently substituted with 0-3 groups selected from halogen, —OR⁶, C₁-C₆ alkyl, C₃-C₇ cycloalkyl, —CN, —COOR⁷, —CONR⁸R⁹, —NR¹⁰C(O)R¹¹, —NR¹²S(O)ₓR¹³, —S(O)ₓNR¹⁴R¹⁵, and —NR¹⁶R¹⁷; wherein R⁶, R⁷, R⁸, R⁹, R¹⁰, R¹¹, R¹², R¹³, R¹⁴, R¹⁵, R¹⁶, R¹⁷ and x are as previously defined in Formula I.

R¹⁸ and R¹⁹ are as previously defined as in Formula IV.

In another embodiment, the compound is represented by Formula VI,

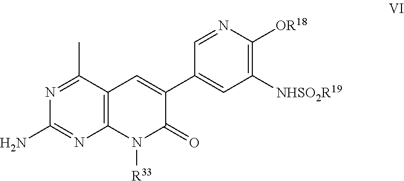

R³³ is C₆-C₁₀ aryl or C₃-C₁₀ heteroaryl, wherein said aryl and heteroaryl are optionally and independently substituted with C₁-C₆ alkyl, C₃-C₇ cycloalkyl, C₂-C₆ heterocyclic alkyl, C₆-C₁₀ aryl, C₃-C₁₀ heteroaryl, C₂-C₆ alkynyl, —CN, —OR, —COOR²⁰, —CONR²¹R²², —NR²³C(O)R²⁴, —NR²⁵S(O)ₘR²⁶, —S(O)ₘNR²⁷R²⁸, or —NR²⁹R³⁰, wherein said C₁-C₆ alkyl, heterocyclic alkyl, C₃-C₇ cycloalkyl, C₂-C₆ alkynyl, and heterocyclyl are further optionally and independently substituted with up to 5 groups selected from halogen, C₁-C₆ alkyl, —CN, —OR, —COOR²⁰, —CONR²¹R²², —NR²³C(O)R²⁴, —NR²⁵S(O)ₘR²⁶, —S(O)ₘNR²⁷R²⁸, and —NR²⁹R³⁰.

R, m, R¹⁸, R¹⁹, R²⁰, R²¹, R²², R²³, R²⁴, R²⁵, R²⁶, R²⁷, R²⁸, R²⁹, R³⁰ are as previously defined as in Formula V.

In further embodiments, some specific examples of the compounds of the invention include:

| | |
|---|---|
| Example | |
| No. | Structure |

| | |
|---|---|
| 1 | 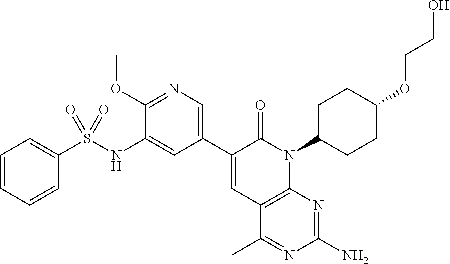 |
| 2 | 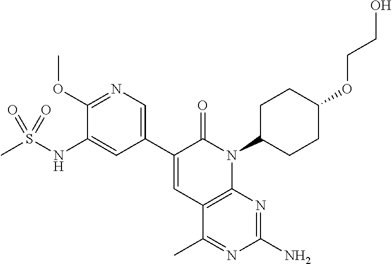 |
| 3 | 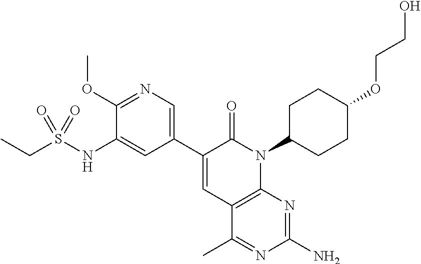 |
| 4 | 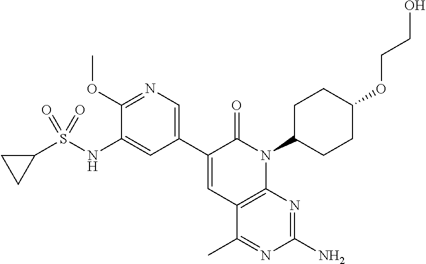 |
| 5 | 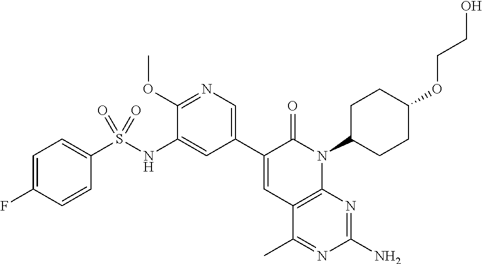 |
| 6 | 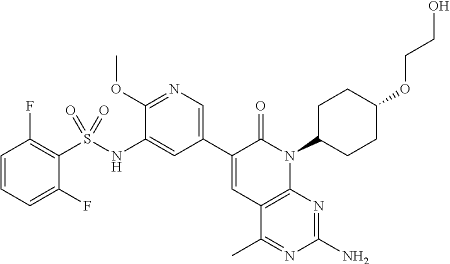 |
| 7 | 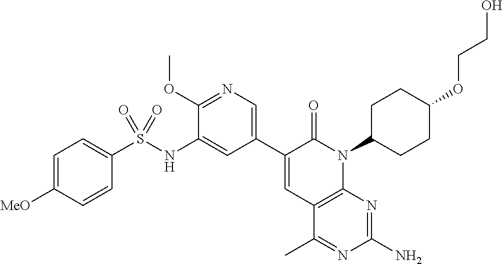 |
| 8 | 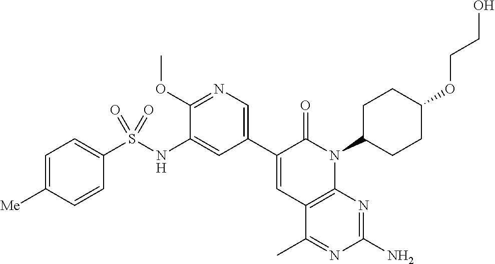 |
| 9 | 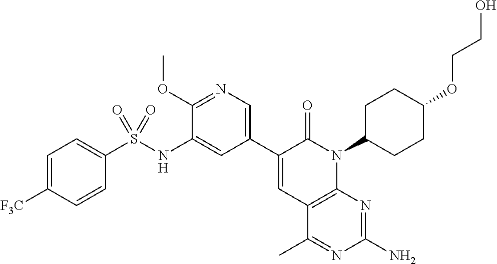 |
| 10 | 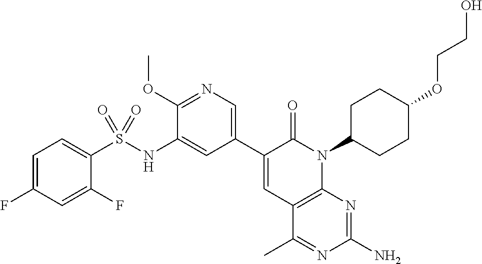 |
| 11 | 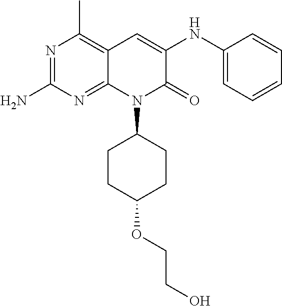 [SMILES: Cc1nc(N)nc2c1cc(Nc1ccccc1)c(=O)n2[C@H]1CC[C@H](OCCO)CC1] |

As used herein except where noted, “alkyl” is intended to include both branched- and P-straight-chain saturated aliphatic hydrocarbon groups having the specified number of carbon atoms. Commonly used abbreviations for alkyl groups are used throughout the specification, e.g. methyl may be represented by conventional abbreviations including “Me” or CH₃ or a symbol that is an extended bond without defined terminal group, e.g.

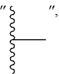

ethyl may be represented by “Et” or CH₂CH₃, propyl may be represented by “Pr” or CH₂CH₂CH₃, butyl may be represented by “Bu” or CH₂CH₂CH₂CH₃, etc. “C₁-C₆ alkyl” (or “C₁-C₆ alkyl”) for example, means linear or branched chain alkyl groups, including all isomers, having the specified number of carbon atoms. C₁₋₆ alkyl includes all of the hexyl alkyl and pentyl alkyl isomers as well as n-, iso-, sec- and t-butyl, n- and isopropyl, ethyl and methyl. If no number is specified, 1-10 carbon atoms are intended for linear or branched alkyl groups. The phrase “C₁-C₆ alkyl, wherein the alkyl group may be unsubstituted or substituted with 1-3 fluorine atoms” refers to alkyl groups having 0, 1, 2 or 3 fluorine atoms attached to one or more carbon atoms. The group “CF₃”, for example, is a methyl group having three fluorine atoms attached the same carbon atom.

The term “cycloalkyl” means carbocycles containing no heteroatoms with 3-7 carbon atoms. Examples of cycloalkyl include cyclopropyl, cyclobutyl, cyclopentyl, cyclohexyl, cycloheptyl, and the like.

“Alkenyl” unless otherwise indicated, means 2-6 carbon chains which contain at least one carbon-carbon double bond, and which may be linear or branched or combinations thereof. Examples of alkenyl include, but are not limited to, vinyl, allyl, isopropenyl, pentenyl, hexenyl, 1-propenyl, 2-butenyl, 2-methyl-2-butenyl, and the like.

The term “alkynyl” refers to a hydrocarbon radical straight, branched or cyclic, containing from 2 to 6 carbon atoms and at least one carbon to carbon triple bond. Up to three carbon-carbon triple bonds may be present. Thus, “C₂-C₆ alkynyl” means an alkynyl radical having from 2 to 6 carbon atoms. Alkynyl groups include ethynyl, propynyl, butynyl, 3-methylbutynyl and so on. The straight, branched or cyclic portion of the alkynyl group may contain triple bonds and may be substituted if a substituted alkynyl group is indicated.

“Aryl” unless otherwise indicated, means mono- and bicyclic aromatic rings containing 6-10 carbon atoms. Examples of aryl include, but are not limited to, phenyl, naphthyl, indenyl and the like. “Aryl” also includes monocyclic rings fused to an aryl group. Examples include tetrahydronaphthyl, indanyl and the like. The preferred aryl is phenyl.

“Heteroaryl” unless otherwise indicated, means a 5- to 10-membered mono- or bicyclic aromatic ring or ring system containing at least 1-5 heteroatoms selected from O, S and N. Examples include, but are not limited to, pyrrolyl, isoxazolyl, isothiazolyl, pyrazolyl, pyridyl, pyridinyl, oxazolyl, oxadiazolyl, thiadiazolyl, thiazolyl, imidazolyl, triazolyl, tetrazolyl, furanyl, triazinyl, thienyl, pyrimidyl, pyrimidinyl, pyridazinyl, pyrazinyl, and the like. Heteroaryl also includes aromatic heterocyclic groups fused to heterocycles that are non-aromatic or partially aromatic, and aromatic heterocyclic groups fused to cycloalkyl rings. Additional examples of heteroaryls include, but are not limited to, indazolyl, thienopyrazolyl, imidazopyridazinyl, pyrazolopyrazolyl, pyrazolopyridinyl, imidazopyridinyl and imidazothiazolyl. Heteroaryl also includes such groups in charged form, e.g., pyridinium. In an embodiment, heteroaryl is oxadiazolyl, pyrazolyl, oxazolyl, pyridinyl and imidazolyl.

“Heterocyclic alkyl”, unless otherwise indicated, means a 3-, 4-, 5-, 6-, 7-, 8-, 9-, or 10-membered mono- or bicyclic saturated ring containing 1-5 heteroatoms selected from N, S and O, in which the point of attachment may be carbon or nitrogen. Examples of “heterocyclic alkyl” include, but are not limited to, azetidinyl, pyrrolidinyl, piperidinyl, piperazinyl, morpholinyl, oxazolidinyl, imidazolidinyl, 2,3-dihydrofuro(2,3-b)pyridyl, benzoxazinyl, and the like. The term also includes partially unsaturated mono- or bicyclic rings that are not aromatic, such as 2- or 4-pyridones attached through the nitrogen or N-substituted-(1H, 3H)-pyrimidine-2,4-diones (N-substituted uracils). Heterocyclic alkyl moreover includes such moieties in charged form, e.g., piperidinium. In an embodiment, heterocyclic alkyl is pyrrolidinyl, piperidinyl, piperazinyl, morpholinyl and oxazolidinyl.

“Halogen (or halo)”, unless otherwise indicated, includes fluorine (fluoro), chlorine (chloro), bromine (bromo) and iodine (iodo). In one embodiment, halo is fluorine or chlorine.

Unless expressly stated to the contrary, substitution by a named substituent is permitted on any atom in a ring (e.g., aryl, a heteroaryl ring, or a saturated heterocyclic ring) provided such ring substitution is chemically allowed and results in a stable compound. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

When any variable (e.g., R, R^{a}, R^{x}, etc.) occurs more than one time in any constituent or in Formula I, its definition on each occurrence is independent of its definition at every other occurrence. Also, combinations of substituents and/or variables are permissible only if such combinations result in stable compounds.

Under standard nomenclature used throughout this disclosure, the terminal portion of the designated side chain is described first, followed by the adjacent functionality toward the point of attachment. For example, a C₁₋₅ alkylcarbonylamino C₁₋₆ alkyl substituent is equivalent to

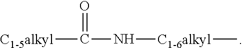

In choosing compounds of the present invention, one of ordinary skill in the art will recognize that the various substituents, i.e. R^{a}, R^{b}, R¹, R², etc., are to be chosen in conformity with well-known principles of chemical structure connectivity and stability.

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Where a substituent or variable has multiple definitions, it is understood that the substituent or variable is defined as being selected from the group consisting of the indicated definitions.

Optical Isomers—Diastereoisomers—Geometric Isomers—Tautomers—Atropisomers:

Compounds of structural Formula I may contain one or more asymmetric centers and can thus occur as racemates and racemic mixtures, single enantiomers, diastereoisomeric mixtures and individual diastereoisomers. The present invention is meant to comprehend all such isomeric forms of the compounds of structural Formula I.

Compounds of structural Formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer or isomers of a compound of the general structural Formula I may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereoisomeric mixture, followed by separation of the individual diastereoisomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

For compounds described herein which contain olefinic double bonds, unless specified otherwise, they are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds of structural Formula I, the atoms may exhibit their natural isotopic abundances, or one or more of the atoms may be artificially enriched in a particular isotope having the same atomic number, but an atomic mass or mass number different from the atomic mass or mass number predominately found in nature. The present invention is meant to include all suitable isotopic variations of the compounds of structural Formula I. For example, different isotopic forms of hydrogen (H) include protium (¹H) and deuterium (²H, also denoted as D). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within structural Formula I, can be prepared without undue experimentation by conventional techniques well known to those skilled in the art or by processes analogous to those described in the Schemes and Examples herein using appropriate isotopically-enriched reagents and/or intermediates. Thus, the present invention covers isotopically-enriched compounds, including deuterated compounds.

The present invention includes all stereoisomeric forms of the compounds of the Formula I. Centers of asymmetry that are present in the compounds of Formula I can all independently of one another have S configuration or R configuration. The invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at the stage of the compounds of the Formula I or at the stage of an intermediate during the synthesis. The present invention also includes all tautomeric forms of the compounds of Formula I.

The present invention includes all atropisomer forms of the compounds of Formula I. Atropisomers are stereoisomers resulting from hindered rotation about single bonds where the steric strain barrier to rotation is high enough to allow for the isolation of the conformers. Atropisomers display axial chirality. Separation of atropisomers is possibly by chiral resolution methods such as selective crystallization.

Salts:

It will be understood that, as used herein, references to the compounds of structural Formula I are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, ascorbate, benzenesulfonate, benzoate, bicarbonate, bisulfate, bitartrate, borate, bromide, butyrate, camphorate, camphorsulfonate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, methanesulfonate, mucate, napsylate, nitrate, N-methylglucamine ammonium salt, oleate, oxalate, pamoate (embonate), palmitate, pantothenate, phosphate/diphosphate, polygalacturonate, propionate, salicylate, stearate, sulfate, subacetate, succinate, tannate, tartrate, teoclate, thiocyanate, tosylate, triethiodide, valerate and the like. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, dicyclohexyl amines and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as O-acetyl, O-pivaloyl, O-benzoyl, and O-aminoacyl, can be employed. Included are those esters and acyl groups known in the art for modifying the solubility or hydrolysis characteristics for use as sustained-release or prodrug formulations.

Some of the compounds of the instant invention may form solvates with water or common organic solvents. Solvates, and in particular, the hydrates of the compounds of the structural formulas described herein are also included in the present invention.

If the compounds of Formula I simultaneously contain acidic and basic groups in the molecule the invention also includes, in addition to the salt forms mentioned, inner salts or betaines (zwitterions). Salts can be obtained from the compounds of Formula I by customary methods which are known to the person skilled in the art, for example by combination with an organic or inorganic acid or base in a solvent or dispersant, or by anion exchange or cation exchange from other salts. The present invention also includes all salts of the compounds of Formula I which, owing to low physiological compatibility, are not directly suitable for use in pharmaceuticals but which can be used, for example, as intermediates for chemical reactions or for the preparation of physiologically acceptable salts.

The present invention also relates to processes for the preparation of the compounds of Formula I which are described in the following and by which the compounds of the invention are obtainable.

One aspect of the invention that is of interest relates to a compound in accordance with Formula I or a pharmaceutically acceptable salt, hydrate, isomer, or solvate thereof for use in a method of treatment of the human by therapy.

Another aspect of the invention relates to a compound in accordance with Formula I or a pharmaceutically acceptable salt, hydrate, isomer, or solvate thereof for use as an anti-cancer agent in a human, wherein said cancer known medically as malignant neoplasm, is a broad group of various diseases, all involving unregulated cell growth includes. Examples of cancer include but not limited to, breast cancer, lung cancer, lymphoma, leukemia.

Cancer drugs refer to other active ingredients in their pharmaceutically acceptable salt, hydrate, isomer, or solvate thereof for use as an anti-cancer agent in a human.

Another aspect of the invention that is of interest is a method of treating inflammations in a human patient in need of such treatment comprising administering a therapeutically effective amount of a compound of Formula I or a pharmaceutically acceptable salt, hydrate, isomer, or solvate thereof.

The present invention also relates to pharmaceutical preparations or pharmaceutical compositions which comprise as active component an effective dose of at least one compound of the Formula I, and/or a physiologically acceptable salt, hydrate, isomer, or solvate thereof, and one or more pharmaceutically acceptable carrier substances and/or additives.

Furthermore, the invention that is of interest is a method for inhibiting protein kinase comprising administering a therapeutically effective amount of a compound of Formula I, or a salt, hydrate, isomer, or solvate thereof, or a pharmaceutical composition of above described. The protein kinase above mentioned includes, but is not limited to, PI3K or mTOR.

The compounds of the Formula I and their physiologically acceptable salts or solvates can be administered to animals, preferably to mammals, and in particular to humans, as pharmaceuticals by themselves, in mixtures with one another or in the form of pharmaceutical preparations. The term “patient” includes animals, preferably mammals and especially humans, who use the instant active agents for the prevention or treatment of a medical condition. Administering of the drug to the patient includes both self-administration and administration to the patient by another person.

A therapeutically effective amount is intended to mean that amount of a drug or pharmaceutical agent that will elicit the biological or medical response of a tissue, a system, animal or human that is being sought by a researcher, veterinarian, medical doctor or other clinician.

Furthermore, a subject of the present invention are pharmaceutical preparations (or pharmaceutical compositions) which comprise as active component an effective dose of at least one compound of the Formula I and/or a physiologically acceptable salt thereof and a customary pharmaceutically acceptable carrier, i.e., one or more pharmaceutically acceptable carrier substances and/or additives.

The pharmaceutical compositions according to the invention can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, tinctures, sprays or transdermal therapeutic systems, or the inhalative administration in the form of nasal sprays or aerosol mixtures, or, for example, microcapsules, implants or rods. The preferred administration form depends, for example, on the disease to be treated and on its severity.

The amount of active compound of the Formula I and/or its physiologically acceptable salts in the pharmaceutical preparations normally is from 0.2 to 1000 mg, preferably from 1 to 500 mg, per dose, but depending on the type of the pharmaceutical preparation it can also be higher. The pharmaceutical preparations usually comprise 0.5 to 90 percent by weight of the compounds of the Formula I and/or their physiologically acceptable salts. The preparation of the pharmaceutical preparations can be carried out in a manner known per se. For this purpose, one or more compounds of the Formula I and/or their physiologically acceptable salts, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human or veterinary medicine.

For the production of pills, tablets, sugar-coated tablets and hard gelatin capsules it is possible to use, for example, lactose, starch, for example maize starch, or starch derivatives, talc, stearic acid or its salts, etc. Carriers for soft gelatin capsules and suppositories are, for example, fats, waxes, semisolid and liquid polyols, natural or hardened oils, etc. Suitable carriers for the preparation of solutions, for example of solutions for injection, or of emulsions or syrups are, for example, water, physiologically sodium chloride solution, alcohols such as ethanol, glycerol, polyols, sucrose, invert sugar, glucose, mannitol, vegetable oils, etc. It is also possible to lyophilize the compounds of the Formula I and their physiologically acceptable salts and to use the resulting lyophilisates, for example, for preparing preparations for injection or infusion. Suitable carriers for microcapsules, implants or rods are, for example, copolymers of glycolic acid and lactic acid.

Besides the active compounds and carriers, the pharmaceutical preparations can also contain customary additives, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

The dosage of the active compound of the Formula I to be administered and/or of a physiologically acceptable salt thereof depends on the individual case and is, as is customary, to be adapted to the individual circumstances to achieve an optimum effect. Thus, it depends on the nature and the severity of the disorder to be treated, and also on the sex, age, weight and individual responsiveness of the human or animal to be treated, on the efficacy and duration of action of the compounds used, on whether the therapy is acute or chronic or prophylactic, or on whether other active compounds are administered in addition to compounds of the Formula I. In general, a daily dose of approximately 0.01 to 100 mg/kg, preferably 0.01 to 10 mg/kg, in particular 0.3 to 5 mg/kg (in each case mg per kg of bodyweight) is appropriate for administration to an adult weighing approximately 75 kg in order to obtain the desired results. The daily dose can be administered in a single dose or, in particular when larger amounts are administered, be divided into several, for example two, three or four individual doses. In some cases, depending on the individual response, it may be necessary to deviate upwards or downwards from the given daily dose.

The compounds of the Formula I bind to the mineralocorticoid receptor and antagonize the biological effects of aldosterone and cortisol. On account of this property, apart from use as pharmaceutically active compounds in human medicine and veterinary medicine, they can also be employed as a scientific tool or as aid for biochemical investigations in which such an effect on the mineralocorticoid receptor is intended, and also for diagnostic purposes, for example in the in vitro diagnosis of cell samples or tissue samples. The compounds of the Formula I and salts thereof can furthermore be employed, as already mentioned above, as intermediates for the preparation of other pharmaceutically active compounds.

The above-mentioned compounds are also of use in combination with other pharmacologically active compounds. Additional active compounds that may be used in combination with the compounds of the instant invention, either co-administered or in a fixed combination, include, but are not limited to anticancer alkylating or intercalating agents, antimetabolites, purine antagonists or pyrimidine antagonists, spindle poisons, podophyllotoxins, antibiotics, nitrosoureas, inorganic ions, enzymes, hormones, mTOR inhibitors, protease inhibitors, NF-kB inhibitor, other inhibitors of kinases (e.g. Src, Brc/Abl, kdr, flt3, aurora-2, GSK-3, EGFR, VEGFR, PDGFR, cMET, MEK, AKT, PI3K, c-kit, fit-3, IGFR, ErbB2, etc), antibodies, soluble receptor or other receptor antagonists against a receptor or hormone implicated in a cancer, etc.

Examples of other active ingredients that may be administered in combination with a compound of Formula I, and either administered separately or in the same pharmaceutical composition, include, but are not limited to:

Mechlorethamine, Chlorambucil, Cyclophosphamide, Melphalan, Ifosfamide, Methotrexate, 6-Mercaptopurine, 5-Fluorouracil, Cytarabile, Gemcitabine, Vinblastine, Vincristine, Vinorelbine, Paclitaxel, Etoposide, Irinotecan, Topotecan, Doxorubicin, Bleomycin, Mitomycin, Carmustine, Lomustine, Cisplatin, Carboplatin, Oxaliplatin, Oxiplatin, Asparaginase, Tamoxifen, Leuprolide, Flutamide, Megestrol, Sirolimus, Temsirolimus, Everolimus, AP23573, Velcade, Iressa, Tarceva, Herceptin, Avastin, Erbitux, Zyloprim, Alemtuzmab, Altretamine, Amifostine, Nastrozole, MLN-591, MLN591RL, MLN2704, Arsenic trioxide, Bexarotene, Busulfan, Capecitabine, Gliadel Wafer, Celecoxib, Chloramubucil, Cisplatin-epinephrine gel, Cladribine, Cytarabine liposomal, Daunorubicin liposomal, Daunorubicin, Daunomycin, Dexrazoxane, Docetaxel, Doxorubicin, Elliott's B solution, Epirubicin, Estramustine, Etoposide Phosphate, Etoposide, Exemestane, Fludarabine, 5-FU, Fulvestrant, Gemcitabine, Gemtuzumab-ozogamicin, Goserelin acetate, Hydroxyurea, Idarubicin, Idamycin, Imatinib mesylate, irinotecan, MLN576, Letrozole, Leucovorin, Leucovorin levamisole, melphalan, L-PAM, Mesna, Mitomycin C, Mitoxantrone, Methoxsalen, MLN518, MLN608, Itoxantrone, Rituximab, Talc, Temozolamide, Teniposide, VM-26, Topotecan, Pegademase, Pentostatin, Porfimer sodium, 2C4, Tretinoin, ATRA, Valrubicin, Vinorelbine, Pamidronate, Zoledronate,

The compounds of Formula I can be synthesized in accordance with the general schemes provided below, taking into account the specific examples that are provided. Throughout the synthetic schemes and examples, abbreviations are used with the following meanings unless otherwise indicated:

Ac=acetate, acetyl;

aq. is aqueous;

Ar is Aryl;

Bn is benzyl;

Boc is tertbutylcarbamoyl;

br is broad;

Bu is butyl;

^{t}Bu is tert-butyl;

celite is Celite® diatomaceous earth;

CHO is Chinese hamster ovary

cpm is counts per minute;

cPr is cyclopropyl;

DCM is dichloromethane;

DIBALH is diisobutylaluminium hydride;

DMF is N,N-dimethylformamide;

DMSO is dimethyl sulfoxide;

EDC is N-(3-dimethylaminopropyl)-N′-ethylcarbodiimide hydrochloride;

EDTA is ethylendiamine tetraacetic acid;

ES-MS is electrospray ion-mass spectroscopy;

Et is ethyl;

Et₂O is diethyl ether;

EtOH is ethanol,

EtOAc is ethyl acetate;

halo is a halogen (preferably fluorine or chlorine),

HATU is O-(7-azabenzotriazol-1-yl)-N,N,N′,N′-tetramethyluronium hexafluorophosphate;

HetAr or HAR is Heteroaryl;

¹HNMR is proton nuclear magnetic resonance;

HPLC is high performance liquid chromatography;

Hz is hertz;

i is Iso;

kg is kilogram;

LC/MS is Liquid chromatography/Mass Spectroscopy;

LiHMDS is lithium bis(trimethylsilyl)amide;

M is molar;

Me is methyl;

μg is microgram;

MeCN is acetonitrile;

MeOH is methanol;

MHz is megahertz;

mm is millimeter;

μL is microliter;

mM is milimolar;

μM is micromolar;

mmol is milimoles;

MS is mass spectrum, and a mass spectrum obtained by ES-MS may be denoted herein by “ES”;

mw is microwave;

m/z is mass to charge ratio;

n is normal;

NaHMDS is sodium hexamethyldisilazide;

nm is nanometer;

nPr is n-propyl;

p is para;

Ph is phenyl;

Pr is propyl;

RP HPLC is Reverse Phase High Performance Liquid Chromatography;

rt is room temperature;

sec is secondary;

^{t}Bu is tert-butyl;

^{t}BuOH is tert-butanol;

tert is tertiary;

TFA is trifluoroacetic acid;

THF is tetrahydrofuran;

TLC is thin layer chromatography;

U is units

UV is ultraviolet;

Synthetic methods for preparing the compounds of the present invention are illustrated in the following Examples. Starting materials are commercially available or may be made according to procedures known in the art or as illustrated herein.

### EXAMPLES

### Example 1

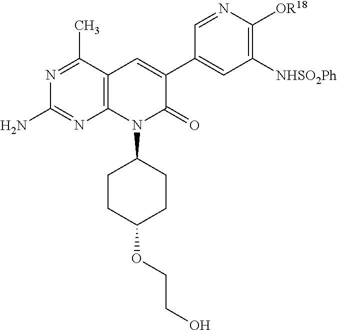

Step 1.

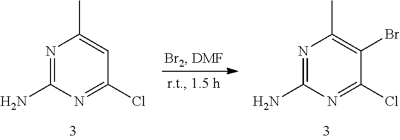

To a mixture of 4-chloro-6-methylpyrimidin-2-amine (2 g, 13.9 mmol) in dichloromethane (100 mL) was added bromine (0.75 mL, 14.6 mmol) slowly. The resulting suspension was stirred at room temperature for 1.5 hours. The reaction was diluted with a mixture of dichloromethane and methanol (10:1, 250 mL) and was washed with saturated sodium bicarbonate (2×150 mL) and brine (150 mL), dried with Na₂SO₄, filtered and concentrated to afford 5-bromo-4-chloro-6-methylpyrimidin-2-amine.

¹H NMR (300 MHz, CDCl₃): ppm 5.219 (s, 2H) 2.515 (s, 3H);

M+H: 223, 224, 226.

Step 2.

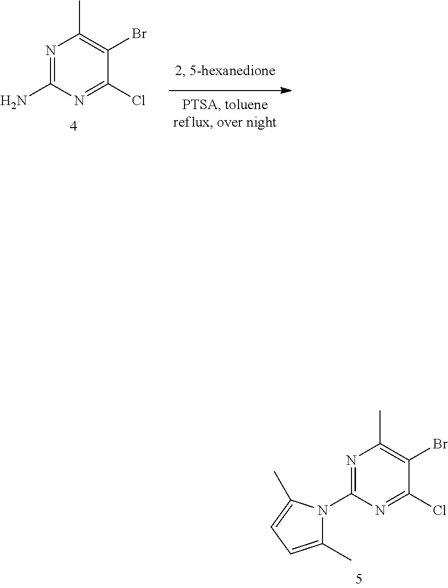

A flask containing a mixture of 5-bromo-4-chloro-6-methylpyrimidin-2-amine (50.0 g, 224.75 mmol), 2,5-hexanedione (40 mL, 338.54 mmol), and p-toluenesulfonic acid (2.14 g, 11.26 mmol) in toluene (1 L) was fitted with a Dean-stark apparatus and a condenser. After refluxing overnight, the solution was cooled to room temperature and concentrated. The crude mixture was purified by flash silica gel chromatography (15/1 hexane/dichloromethane) to afford 5-bromo-4-chloro-2-(2,5-dimethyl-1H-pyrrol-1-yl)-6-methylpyrimidine.

¹H NMR (300 MHz, CDCl₃): ppm 5.904 (s, 2H) 2.719 (s, 3H) 2.389 (s, 6H);

M+H: 300, 302, 304

Step 3.

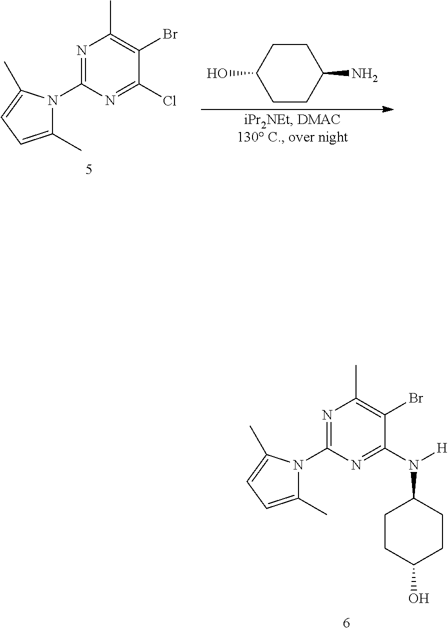

A mixture of 5-bromo-4-chloro-2-(2,5-dimethyl-1H-pyrrol-1-yl)-6-methylpyrimidine (15.0 g, 50.1 mmol), trans-4-aminocyclohexanol hydrochloride (7.5 g, 65.1 mmol), and diisopropylethyl amine (27 mL) indimethylacetamide (75 mL) was heated at 130° C. in a sealed tube overnight. The reaction mixture was diluted with water. The aqueous layer was separated and extracted with ether (2×250 mL). The combined organic layers were washed with brine, dried with Na₂SO₄, filtered and concentrated. The crude product was purified by flash silica gel chromatography (¼ ethyl acetate/hexane) to afford trans-4-((5-bromo-2-(2,5-dimethyl-1H-pyrrol-1-yl)-6-methylpyrimidin-4-yl)amino)cyclohexanol.

¹H NMR (500 MHz, CDCl₃): ppm 5.870 (s, 2H) 5.335 (d, 1H) 4.00 (t, 1H) 3.602 (s, 1H) 2.517 (s, 3H) 2.365 (s, 6H) 2.100 (d, 2H) 1.980 (d, 2H) 1.28-1.43 (m, 4H);

M+H: 379, 381

Step 4.

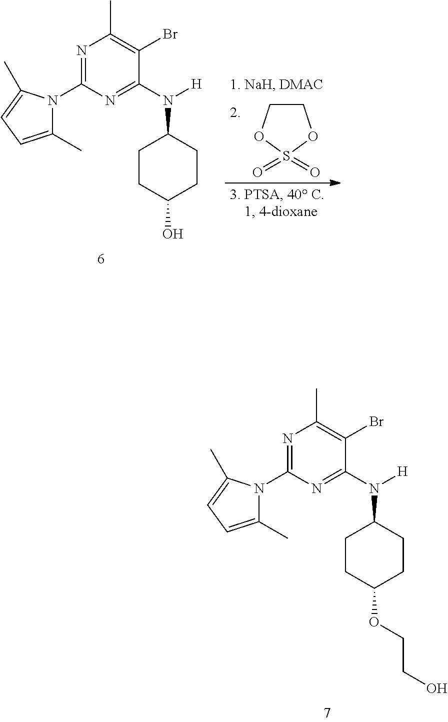

To a cooled (0° C.) solution of trans-4-((5-bromo-2-(2,5-dimethyl-1H-pyrrol-1-yl)-6-methylpyrimidin-4-yl)amino)cyclohexanol (1.0 g, 2.65 mmol) in dimethylacetamide (15 mL) was added sodium hydride (60% dispersion in oil, 0.19 g, 7.96 mmol) portion-wise. After 2.5 hours, 1,3,2-dioxathiolane-2,2-dioxane (0.48 g, 3.98 mmol) was added portion-wise (0.25 eq. each time). The mixture was stirred at room temperature overnight, quenched with methanol and concentrated. The residue was diluted with 1, 4 dioxane (100 mL) and water (3 mL). p-Toluenesulfonicacid (0.685 g, 3.98 mmol) was added and the mixture was heated at 40° C. for 3 hours. Additional p-toluenesulfonic acid (0.685 g, 3.98 mmol) was added to the mixture and the resulting mixture was heated at 40° C. for another 3 hours. More p-toluenesulfonic acid (0.91 g, 5.3 mmol) was added and the mixture was heated at 40° C. overnight. The reaction mixture was cooled to room temperature and slowly quenched with a solution of sodium bicarbonate (4 g) in water (20 mL). The mixture was concentrated and the residue was extracted with ethylacetate. The combined organic layers were dried with Na₂SO₄, filtered and concentrated. The crude product was purified by flash silica gel chromatography (20/1 to 10/1 ethyl acetate/hexane) to afford 2-((trans-4-((5-bromo-2-(2,5-dimethyl-1H-pyrrol-1-yl)-6-methylpyrimidin-4-yl)amino) cyclohexyl)oxy)ethanol.

¹H NMR (500 MHz, CDCl₃): ppm 5.862 (s, 2H) 5.32 (d, 1H) 4.03 (t, 1H) 3.72 (t, 2H) 3.59 (t, 2H) 3.34 (t, 1H) 2.51 (s, 3H) 2.36 (s, 6H) 2.05-2.15 (m. 5H) 1.21-1.45 (m. 6H);

M+H: 424

Step 5.

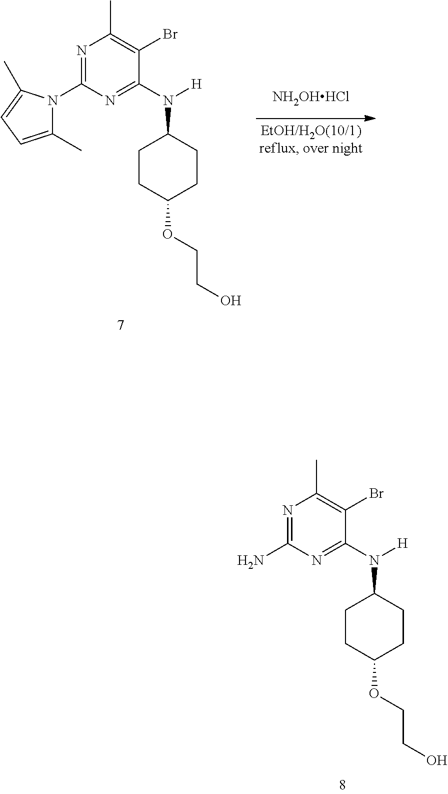 [SMILES: Cc1nc(-n2c(C)ccc2C)nc(N[C@H]2CC[C@H](OCCO)CC2)c1Br ; "NH2OH·HCl" ; "EtOH/H2O(10/1)reflux, over night" ; Cc1nc(N)nc(N[C@H]2CC[C@H](OCCO)CC2)c1Br]

2-((trans-4-((5-bromo-2-(2,5-dimethyl-1H-pyrrol-1-yl)-6-methylpyrimidin-4-yl)amino)cyclohexyl)oxy)ethanol (1 g, 2.36 mmol) and hydroxylamine hydrochloride was diluted with a mixture of ethanol and water (10:1, 20 mL), and then the resulting mixture was heated to reflux for overnight. The reaction mixture was concentrated and the residue was basified with 50% saturated sodium bicarbonate. The aqueous mixture was extracted with dichloromethane and the combined organics were dried with Na₂SO₄, filtered, and concentrated. The crude product was purified by flash silica gel chromatography (½ EA/hexane) to afford 2-(trans-4-(5-bromo-2-(2,5-dimethyl-1H-pyrrol-1-yl)-6-methylpyrimidin-4-ylamino) cyclohexyloxy)ethanol.

¹H NMR (500 MHz, CDCl₃): ppm 5.094 (d, 1H) 4.840 (s, 2H) 3.879-3.909 (m, 1H) 3.732 (t, 2H) 3.593 (t, 2H) 3.307-3.348 (m, 1H) 2.327 (s, 3H) 2.100 (t, 5H) 1.40-1.46 (m, 2H) 1.234-1.287 (m, 3H);

M+H: 345, 347

Step 6.

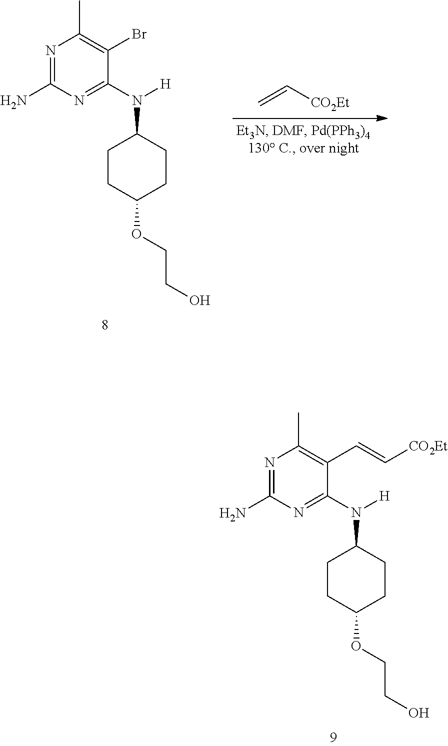

In a sealed tube was added 2-(trans-4-(5-bromo-2-(2,5-dimethyl-1H-pyrrol-1-yl)-6-methylpyrimidin-4-ylamino)cyclohexyloxy)ethanol (3.5 g, 10.2 mmol), ethyl acrylate (2.2 mL, 20.4 mmol), and triethylamine (50 mL). The mixture was bubbled with argon for 10 minutes then tetrakis(triphenylphosphin)-palladium (0) (1.17 g, 1.02 mmol) was added. The vial was sealed and the reaction was heated to 130° C. for overnight. The reaction was cooled to room temperature and concentrated. The crude product was purified by flash silica gel chromatography (1/1 EA/hexane) to afford (E)-ethyl 3-(2-amino-4-(trans-4-(2-hydroxyethoxy)cyclohexylamino)-6-methyl pyrimidin-5-yl)acrylate.

¹HNMR (500 MHz, CDCl₃): ppm 7.676 (d, 1H) 6.035 (d, 1H) 4.916 (s, 3H) 4.244-4.287 (m, 2H) 3.974-4.003 (m, 1H) 3.734 (t, 2H) 3.595 (t, 2H) 3.493 (s, 1H) 3.312-3.328 (m, 1H) 2.318 (s, 3H) 2.068-2.141 (m, 5H) 1.392-1.458 (m, 3H) 1.341 (t, 3H) 1.189-1.261 (m, 3H);

M+H: 365

Step 7.

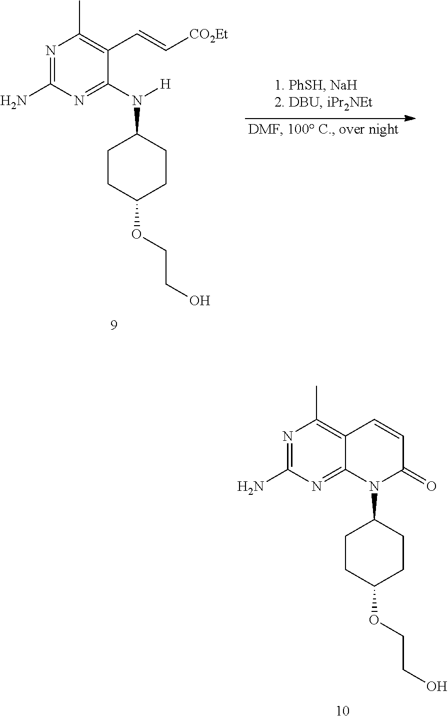 [SMILES: CCOC(=O)/C=C/c1c(C)nc(N)nc1N[C@H]1CC[C@H](OCCO)CC1 ; "1. PhSH, NaH2. DBU, iPr2NEt" ; "DMF, 100° C., over night" ; Cc1nc(N)nc2c1ccc(=O)n2[C@H]1CC[C@H](OCCO)CC1]

To a cooled (0° C.) solution of thiophenol (0.36 g, 3.3 mmol) in dimethylformamide (15 mL) was added sodium hydride (60% dispersion in oil, 66 mg, 1.65 mmol) portionwise. The mixture was stirred until no gas was formed. Then (E)-ethyl 3-(2-amino-4-(trans-4-(2-hydroxyethoxy)cyclohexylamino)-6-methylpyrimidin-5-yl)acrylate (0.6 g, 1.65 mmol), 1,8-diazabicyclo(5,4,0)undec-7-ene (0.96 mL, 6.6 mmol), and diisopropylethyl amine (1.74 mL, 9.9 mmol) were added. Then the reaction was heated to 100° C. for overnight. The reaction mixture was cooled to room temperature and concentrated. The residue was purified by flash silica gel chromatography (1:1 EtOAc/hexane) to afford 2-amino-8-(trans-4-(2-hydroxyethoxy)cyclohexyl)-4-methylpyrido-[2,3-d]pyrimidin-7(8H)-one.

¹HNMR (500 MHz, CDCl₃): 7.636 (d, 1H) 6.341 (d, 1H) 5.436 (s, 1H) 5.169 (s, 2H) 3.743 (t, 2H) 3.622-3.649 (m, 2H) 3.425-3.485 (m, 1H) 2.795 (d, 2H) 2.547 (s, 3H) 2.176 (d, 2H) 1.407-1.474 (m, 2H) 1.207-1.256 (m, 2H);

M+H: 319

Step 8.

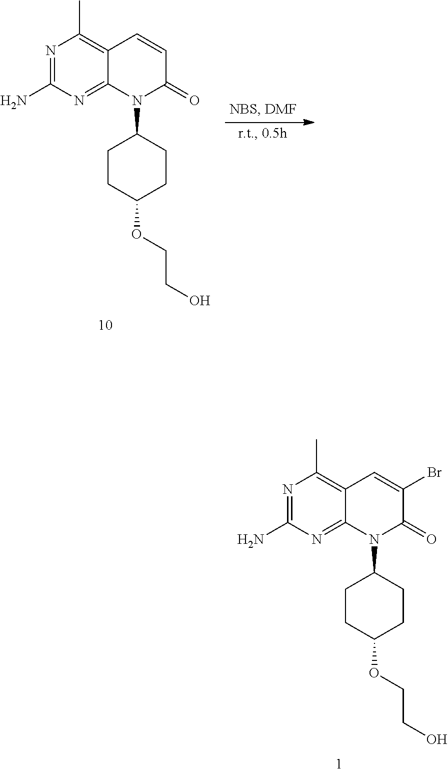

To a solution of 2-amino-8-(trans-4-(2-hydroxyethoxy)cyclohexyl)-4-methylpyrido[2,3-d]pyrimidin-7(8H)-one (1.2 g, 3.77 mmol) in dimethylformamide (25 mL) was added N-bromosuccinimide (0.73 g, 4.15 mmol). After stirring for 30 min at room temperature the solution was diluted with water and extracted with ethyl acetate. The combined organics were dried with Na₂SO₄, filtered, and concentrated. The crude product was purified by flash silica gel chromatography (0-5% DCM/methanol) to afford 2-amino-6-bromo-8-(trans-4-(2-hydroxyethoxy)cyclohexyl)-4-methylpyrido[2,3-d]pyrimidin-7(8H)-one.

¹HNMR (300 MHz, CDCl₃):ppm 8.095 (s, 1H) 5.506 (s, 1H) 5.232 (s, 2H) 3.731-3.761 (m, 2H) 3.625 (t, 2H) 3.397-3.497 (m, 1H) 2.782 (s, 2H) 2.563 (s, 3H) 2.189 (d, 2H) 1.669 (d, 2H) 1.444 (d, 2H);

M+H: 397, 399

Step 9.

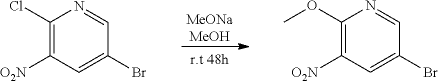

In a 25 ml single-port round bottom flask were added 2-chloro-3-nitro-5-bromo pyridine (500 mg, 2.11 mmol) and methanol. The solution of sodium methoxide (228 mg, 4.22 mmol) in methanol was added into the flask. The reaction mixture was stirred at room temperature for 48 hours. The reaction was quenched with water and concentrated. The residue was extracted with ethyl acetate, dried with Na₂SO₄, and filtered to afford product 2-methoxy-3-nitro-5-bromo pyridine as a light yellow solid.

¹HNMR (300 MHz, CDCl₃): 8.502 (d, 1H) 8.302 (d, 1H) 4.113 (s, 3H);

M+H: 233, 235

Step 10.

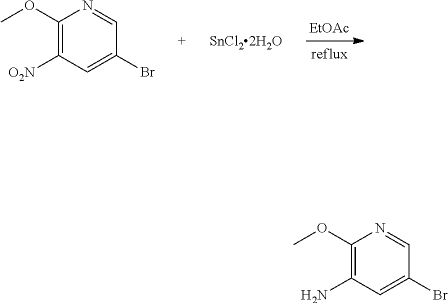

To a solution of 2-methoxy-3-nitro-5-bromo pyridine (400 mg, 1.7 mmol) in EtOAc was added SnCl₂.2H₂O. This reaction mixture was heated to reflux overnight. Then the mixture was concentrated and the residue was dissolved in 2 mol/L sodium hydroxide solution and extracted with DCM for three times. The combined organics were dried with Na₂SO₄, filtered through diatomite and concentrated to afford the crude product 2-methoxy-3-amino-5-bromo pyridine (350 mg). It was used for the next step without any further purification.

¹HNMR (300 MHz, CDCl₃): 7.586 (d, 1H) 6.981 (d, 1H) 3.961 (s, 3H) 3.848 (d, 2H);

M+H: 203, 205

Step 11.

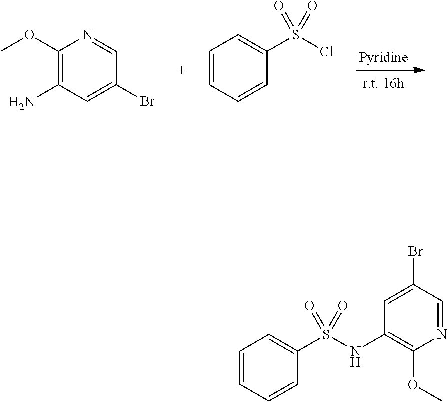

In a 25 ml round bottom flask was added 2-methoxy-3-amino-5-bromo pyridine and pyridine. Then the solution of benzenesulfonyl chloride in pyridine was added into the flask dropwise. The reaction mixture was stirred at room temperature for 16 hours. Then the mixture was concentrated, the residue was diluted with EtOAc and water. The organic layer was dried with Na₂SO₄, filtered and concentrated. The residue was purified by flash silica gel chromatography (1:25 EtOAc:hexane) to afford the brown solid product N-(5-bromo-2-methoxypyridin-3-yl)benzenesulfonamide.

¹HNMR (300 MHz, CDCl₃): 7.880-7.904 (m, 2H) 7.814 (t, 2H) 7.576 (d, 1H) 7.487 (t, 2H) 6.924 (s, 1H) 3.810 (s, 3H);

M+H: 343, 345

Step 12.

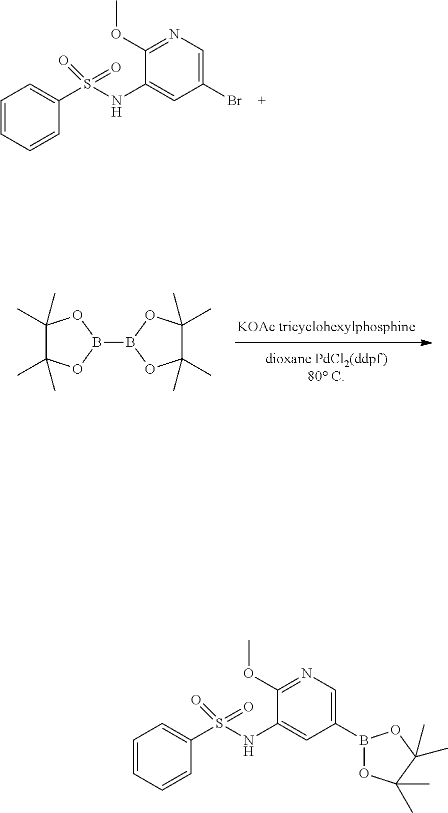

In a 25 ml single-port round bottom flask was added N-(5-bromo-2-methoxypyridin-3-yl)benzenesulfonamide (50 mg, 0.15 mmol), 4,4,4′,4′,5,5,5′,5′-octamethyl-2,2′-bi(1,3,2-dioxaborolane) (41 mg, 0.17 mmol), potassium acetate (21 mg, 0.23 mmol), tricyclohexylphosphine (5 mg, 0.015 mmol), PdCl₂(dppf) (17 mg, 0.02 mmol). The mixture was bubbled with argon. Then 1,4-dioxane was added and the reaction mixture was stirred at 80° C. for 8 hours. LC-MS showed the reaction was complete. The reaction mixture was then filtered through diatomite and concentrated to afford the crude product N-(2-methoxy-5-(4,4,5,5-tetramethyl-1,3,2-dioxaborolan-2-yl)pyridin-3-yl)benzenesulfonami de. It was used for the next step without any further purification.

M+H: 391

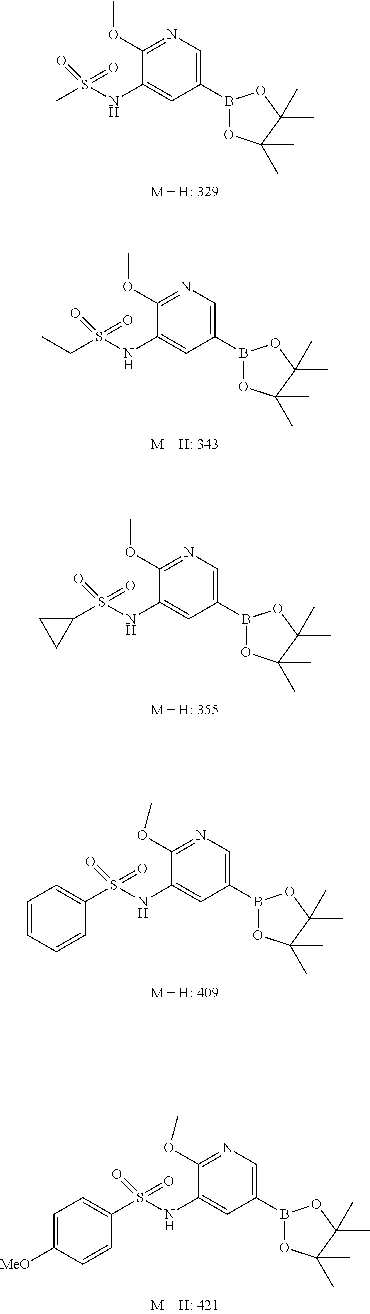

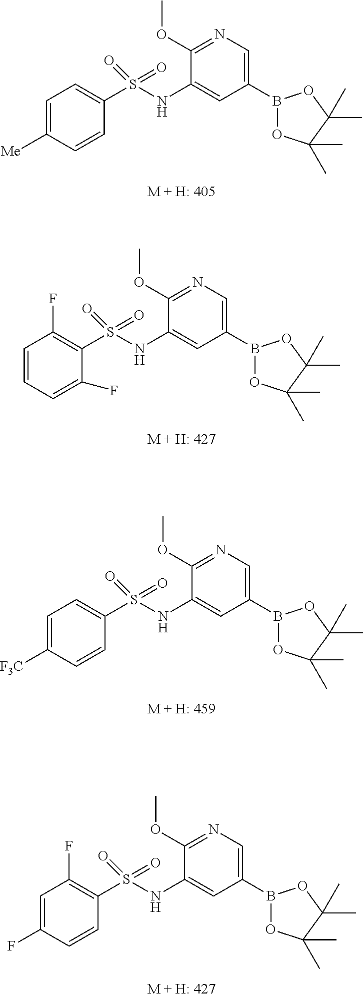 [SMILES: COc1ncc(B2OC(C)(C)C(C)(C)O2)cc1NS(=O)(=O)c1ccc(C)cc1 ; COc1ncc(B2OC(C)(C)C(C)(C)O2)cc1NS(=O)(=O)c1c(F)cccc1F ; COc1ncc(B2OC(C)(C)C(C)(C)O2)cc1NS(=O)(=O)c1ccc(C(F)(F)F)cc1 ; COc1ncc(B2OC(C)(C)C(C)(C)O2)cc1NS(=O)(=O)c1ccc(F)cc1F]

The preparation of the borate esters above all followed the procedure described in steps 11 and 12.

Step 13.

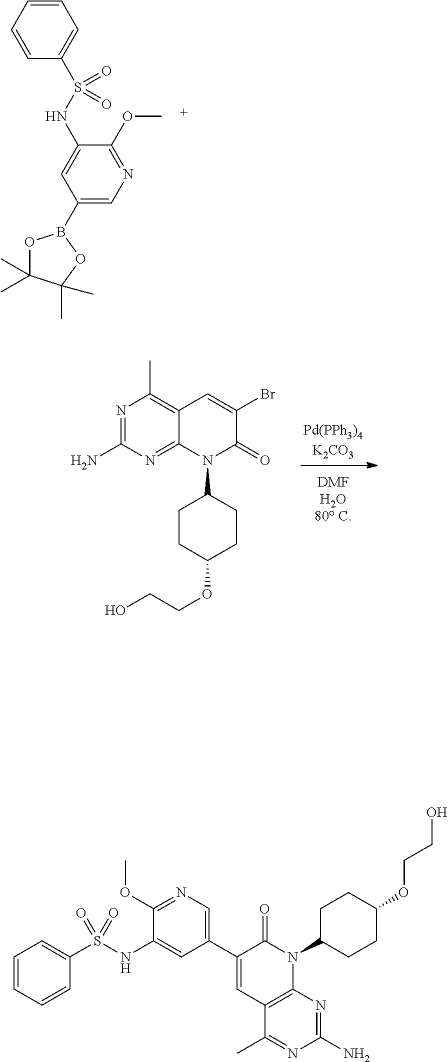

In a 25 ml round bottom flask was added the borate ester product obtained in the last step, 2-amino-6-bromo-8-(trans-4-(2-hydroxyethoxy)cyclohexyl)-4-methylpyrido[2,3-d]pyrimidin-7(8H)-one (59.6 mg, 0.15 mmol), Pd(PPh3)₄ (17 mg, 0.015 mmol) potassium carbonate (62.4 mg, 0.45 mmol). The mixture was bubbled with argon. Then DMF (5 ml) and water (5 ml) were added. The reaction mixture was stirred at 95° C. for 8 hours and the reaction was complete by LC-MS. Then the reaction solution was filtered through diatomite, the filtrate was diluted with water and EA. The aqueous layer was extracted with EtOAc for three times. The combined organics were dried with Na₂SO₄, filtered and concentrated to afford an oily product. It was purified by chromatography (1:2 EtOAc:hexane) to afford the gray solid product N-(5-(2-amino-8-(trans-4-(2-hydroxyethoxy)-cyclohexyl)-4-methyl-7-oxo-7,8-dihydropyrido[2,3-d]pyrimidin-6-yl)-2-methoxypyridin-3-yl)benzenesulfonamide (Example 1).

¹HNMR (300 MHz, CDCl₃): 8.093 (t, 2H) 7.875 (d, 2H) 7.770 (s, 1H) 7.561 (d, 1H) 7.474 (t, 2H) 6.907 (s, 1H) 5.516 (s, 1H) 5.545 (d, 2H) 3.813 (s, 3H) 3.753 (s, 2H) 3.642 (t, 2H) 3.480 (d, 2H) 2.836 (d, 2H) 2.605 (s, 3H) 2.155 (d, 4H) 1.457-1.731 (m, 11H);

M+H: 581

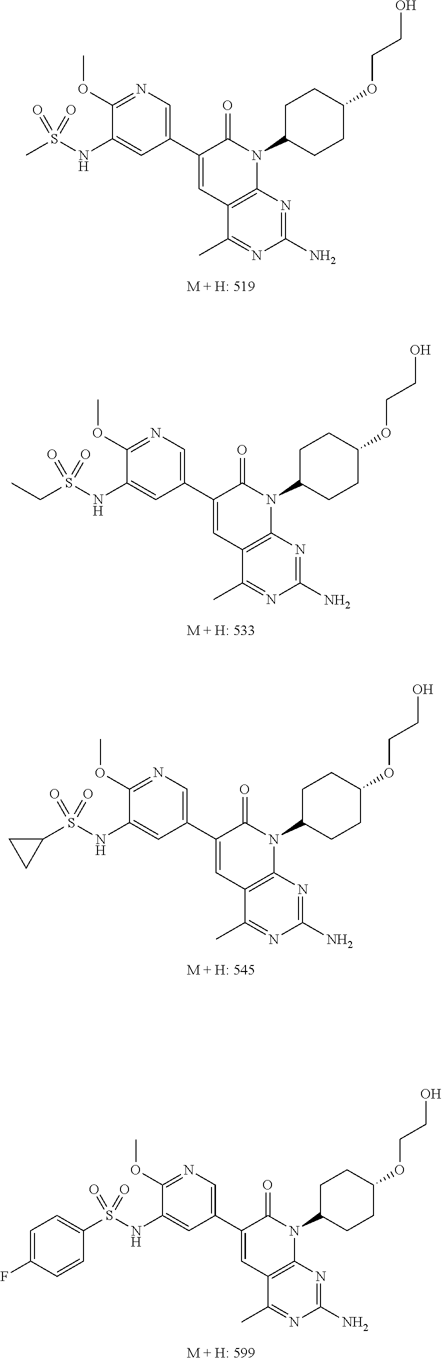

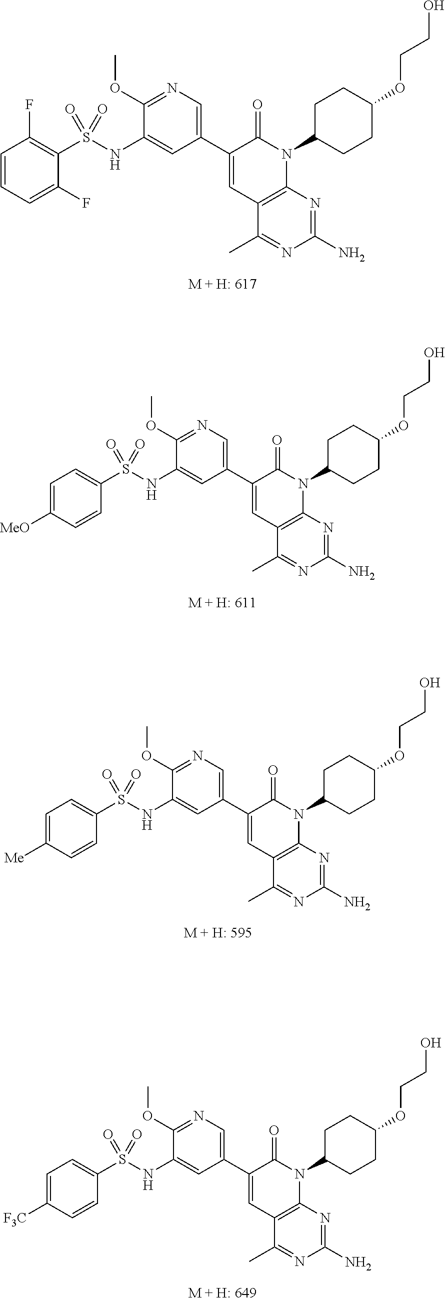

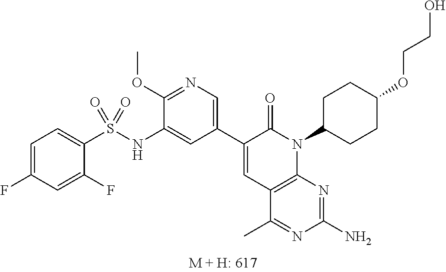

### Example 2-10

The preparation of the compounds above followed the identical Suzuki cross-coupling reaction procedure described in step 13 for Example 1 using the corresponding boronic esters prepared by the protocol described in step 11 and 12 for Example 1.

### Example 2

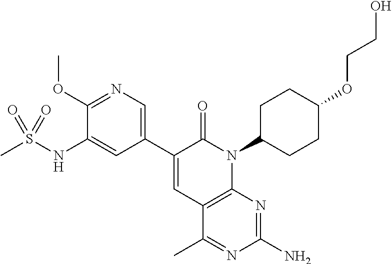

N-(5-(2-amino-8-(trans-4-(2-hydroxyethoxy)cyclohexyl)-4-methyl-7-oxo-7,8-dihydropyrido[2,3-d]pyrimidin-6-yl)-2-methoxypyridin-3-yl)methanesulfonamide.

¹H NMR (400 MHz, CDCl₃): δ 8.16 (s, 1H), 8.06 (s, 1H), 7.75 (s, 1H), 6.90-6.70 (br. s, 1H), 5.40-5.60 (br. s, 2H), 5.32 (s, 3H), 5.25 (s, 1H), 4.15-4.13 (t, J=7.4 Hz, 1H), 4.06 (s, 3H), 3.76 (s, 2H), 3.65-3.64 (d, J=3.6 Hz, 2H), 3.46 (s, 1H), 3.09 (s, 3H), 2.61 (s, 3H), 2.21-2.19 (d, J=8.8 Hz, 2H), 1.48-1.45 (d, J=11.6 Hz, 2H).

M+H: 519

### Example 3

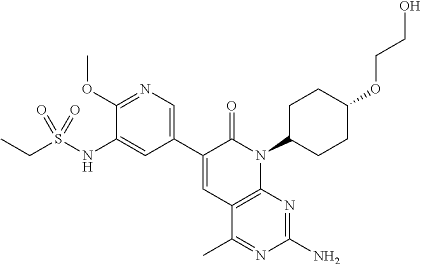

N-(5-(2-amino-8-(trans-4-(2-hydroxyethoxy)cyclohexyl)-4-methyl-7-oxo-7,8-dihydropyrido [2,3-d]pyrimidin-6-yl)-2-methoxypyridin-3-yl)ethanesulfonamide.

¹H NMR (500 MHz, CDCl₃): δ 8.15-8.14 (d, J=1.5 Hz, 1H), 8.04 (s, 1H), 7.72 (s, 1H), 6.70 (s, 1H), 5.40-5.60 (br. s, 2H), 5.24 (s, 2H), 5.18 (s, 1H), 4.15-4.10 (m, 1H), 4.05 (s, 3H), 3.74 (s, 2H), 3.63-3.62 (t, J=4.5 Hz, 2H), 3.47-3.42 (m, 1H), 3.20-3.15 (q, J₁=7.5 Hz, J₂=15 Hz, 2H), 2.81 (s, 2H), 2.59 (s, 3H), 2.19-2.17 (d, J=11 Hz, 2H), 1.71-1.69 (d, J=11 Hz, 2H), 1.49-1.45 (t, J=10.5 Hz, 2H), 1.40-1.37 (t, J=7.5 Hz, 3H).

M+H: 533

### Example 4

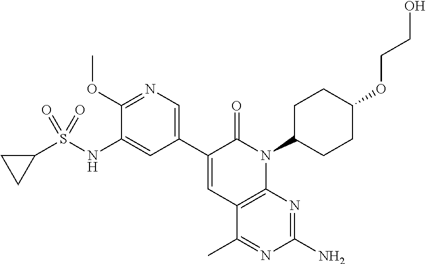

N-(5-(2-amino-8-(trans-4-(2-hydroxyethoxy)cyclohexyl)-4-methyl-7-oxo-7,8-dihydropyrido[2,3-d]pyrimidin-6-yl)-2-methoxypyridin-3-yl)cyclopropanesulfonamide.

¹H NMR (500 MHz, CDCl₃): δ 8.15-8.14 (d, J=1.5 Hz, 1H), 8.08 (s, 1H), 7.73 (s, 1H), 6.72-6.68 (br. s, 1H), 5.55-5.40 (br. s, 2H), 5.30 ((s, 2H), 5.16 ((s, 1H), 4.15-4.10 (q, J₁=7.0 Hz, J₂=14.5 Hz, 2H), 4.05 (s, 3H), 3.74 (s, 2H), 3.64-3.62 (t, J=4.5 Hz, 2H), 3.46-3.45 (m, 1H), 2.81 (m, 2H), 2.60 (s, 3H), 2.17 (s, 3H), 2.08 (s, 1H), 1.72-1.65 (t, J=13.5 Hz, 4H), 1.50-1.45 (t, J=10.5 Hz, 4H).

M+H: 545

### Example 5

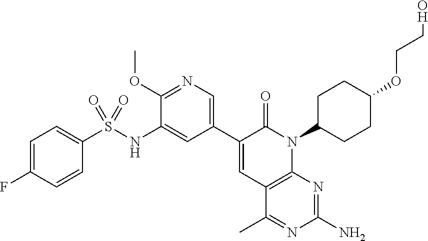

N-(5-(2-amino-8-(trans-4-(2-hydroxyethoxy)cyclohexyl)-4-methyl-7-oxo-7,8-dihydropyrido[2,3-d]pyrimidin-6-yl)-2-methoxypyridin-3-yl)-4-fluorobenzenesulfonamide.

¹H NMR (400 MHz, CDCl₃): δ 8.10 (s, 1H), 8.05 (s, 1H), 7.91-7.88 (t, J=6.2 Hz, 2H), 7.69 (s, 1H), 7.15-7.11 (t, J=8.0 Hz, 2H), 5.60-5.40 (br. s, 1H), 5.26 (s, 2H), 3.83 (s, 3H), 3.74-3.73 (d, J=3.2 Hz, 2H), 3.64-3.63 (d, J=3.2 Hz, 2H), 3.47-3.42 (t, J=10.2 Hz, 1H), 2.83-2.81 (d, J=7.6 Hz, 2H), 2.21-2.18 (d, J=11.2 Hz, 2H), 1.71-1.69 (d, J=10.4 Hz, 2H), 1.50-1.42 (q, J₁=8.0 Hz, J₂=20.0 Hz, 2H).

M+H: 599

### Example 6

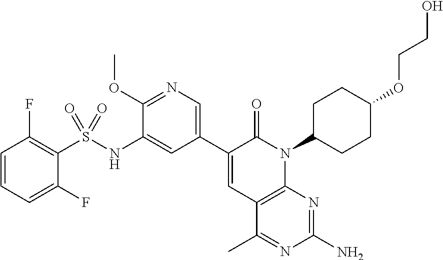

N-(5-(2-amino-8-(trans-4-(2-hydroxyethoxy)cyclohexyl)-4-methyl-7-oxo-7,8-dihydropyrido[2,3-d]pyrimidin-6-yl)-2-methoxypyridin-3-yl)-2,6-difluorobenzenesulfonamide.

¹H NMR (400 MHz, CDCl₃): δ 8.17 (s, 1H), 8.02 (s, 1H), 7.67 (s, 1H), 7.55-7.35 (m, 2H), 7.00-6.96 (t, J=8.8 Hz, 1H), 7.15-7.11 (t, J=8.0 Hz, 2H), 5.60-5.40 (br. s, 1H), 5.29 (s, 2H), 3.92-3.91 (d, J=4.8 Hz, 3H), 3.73 (s, 2H), 3.61 (s, 2H), 3.42 (s, 1H), 2.81 (s, 3H), 2.58-2.57 (d, J=5.2 Hz, 3H), 2.19-2.16 (d, J=10.8 Hz, 2H), 1.45-1.42 (d, J=11.2 Hz, 2H).

M+H: 617

### Example 7

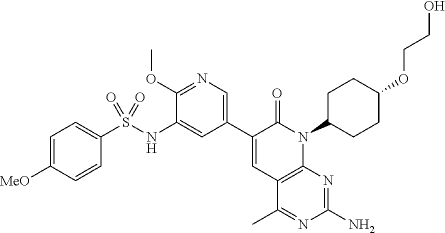

N-(5-(2-amino-8-(trans-4-(2-hydroxyethoxy)cyclohexyl)-4-methyl-7-oxo-7,8-dihydropyrido[2,3-d]pyrimidin-6-yl)-2-methoxypyridin-3-yl)-4-methoxybenzenesulfonamide.

¹H NMR (500 MHz, CDCl₃): δ 8.08 (s, 2H), 7.82-7.80 (d, J=10.0 Hz, 2H), 7.69 (s, 1H), 6.93-6.91 (d, J=10.0 Hz, 2H), 5.60-5.40 (br. s, 1H), 5.28 (s, 2H), 3.88 (s, 3H), 3.85 (s, 3H), 3.75-3.73 (t, J=4.3 Hz, 2H), 3.65-3.63 (t, J=4.3 Hz, 2H), 3.48-3.44 (t, J=11.3 Hz, 1H), 2.89 (m, 1H), 2.60 (s, 3H), 2.22-2.19 (d, J=15.0 Hz, 2H), 1.73-1.71 (d, J=5.0 Hz, 2H), 1.59 (s, 2H), 1.49-1.46 (d, J=15.0 Hz, 2H).

M+H: 611

### Example 8

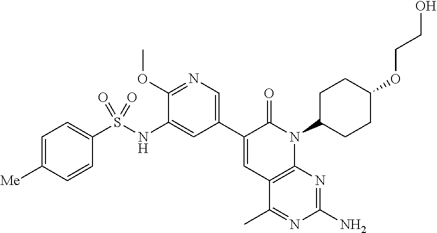

N-(5-(2-amino-8-(trans-4-(2-hydroxyethoxy)cyclohexyl)-4-methyl-7-oxo-7,8-dihydropyrido[2,3-d]pyrimidin-6-yl)-2-methoxypyridin-3-yl)-4-methylbenzenesulfonamide.

¹H NMR (500 MHz, CDCl₃): δ 8.08 (s, 2H), 7.77-7.75 (d, J=7.5 Hz, 2H), 7.69 (s, 1H), 7.27 (s, 1H), 6.87-6.84 (t, J=8.0 Hz, 1H), 5.60-5.40 (br. s, 1H), 5.25 (s, 2H), 3.84 (s, 3H), 3.76-3.74 (t, J=4.3 Hz, 2H), 3.65-3.63 (t, J=4.3 Hz, 2H), 3.48-3.44 (t, J=11.3 Hz, 1H), 2.85 (s, 2H), 2.60 (s, 3H), 2.39 (s, 3H), 2.22-2.20 (d, J=5.0 Hz, 2H), 1.73-1.71 (d, J=5.0 Hz, 2H), 1.62 (s, 2H), 1.51-1.49 (d, J=5.0 Hz, 2H).

M+H: 595

### Example 9

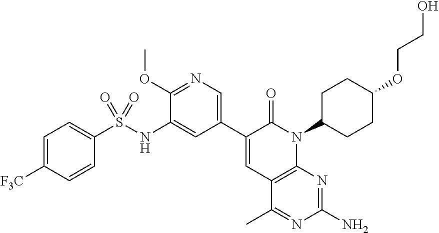

N-(5-(2-amino-8-(trans-4-(2-hydroxyethoxy)cyclohexyl)-4-methyl-7-oxo-7,8-dihydropyrido [2,3-d]pyrimidin-6-yl)-2-methoxypyridin-3-yl)-4-(trifluoromethyl)benzenesulfonamide.

¹H NMR (400 MHz, CDCl₃): δ 8.16-8.14 (d, J=10.4 Hz, 1H), 8.07-8.06 (d, J=2 Hz, 1H), 8.02-8.00 (d, J=8.4 Hz, 2H), 7.75-7.73 (d, J=8.4 Hz, 2H), 7.71 (s, 1H), 5.60-5.40 (br. s, 1H), 5.31 ((s, 2H), 3.80 (s, 3H), 3.76-3.74 (t, J=4.6 Hz, 3H), 3.65-3.61 (q, J₁=9.2 Hz, J₂=4.8 Hz, 2H), 3.48-3.42 (m, 1H), 2.88-2.80 (m, 2H), 2.59 (s, 1H), 2.22-2.19 (d, J=10 Hz, 2H), 1.72-1.70 (d, J=10.8 Hz, 2H), 1.48-1.45 (d, J=12.8 Hz, 2H).

M+H: 649

### Example 10

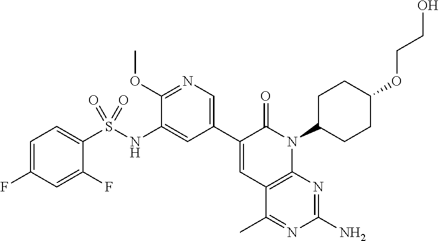

N-(5-(2-amino-8-(trans-4-(2-hydroxyethoxy)cyclohexyl)-4-methyl-7-oxo-7,8-dihydropyrido[2,3-d]pyrimidin-6-yl)-2-methoxypyridin-3-yl)-2,4-difluorobenzenesulfonamide.

¹H NMR (300 MHz, CDCl₃): δ 8.08-8.07 (d, J=2.1 Hz, 1H), 8.02-8.01 (d, J=1.8 Hz, 1H), 7.95-7.87 (m, 1H), 7.68 (s, 1H), 6.97-6.91 (m, 2H), 5.60-5.40 (br. s, 1H), 5.29 ((s, 2H), 3.91 (s, 3H), 3.73 (s, 2H), 3.65-3.62 (t, J=4.5 Hz, 2H), 3.48-3.41 (m, 1H), 2.65-2.90 (m, 2H), 2.59 (s, 3H), 2.22-2.19 (m, 2H), 1.80-1.61 (m, 3H), 1.52-1.41 (m, 2H).

M+H: 617

### Example 11

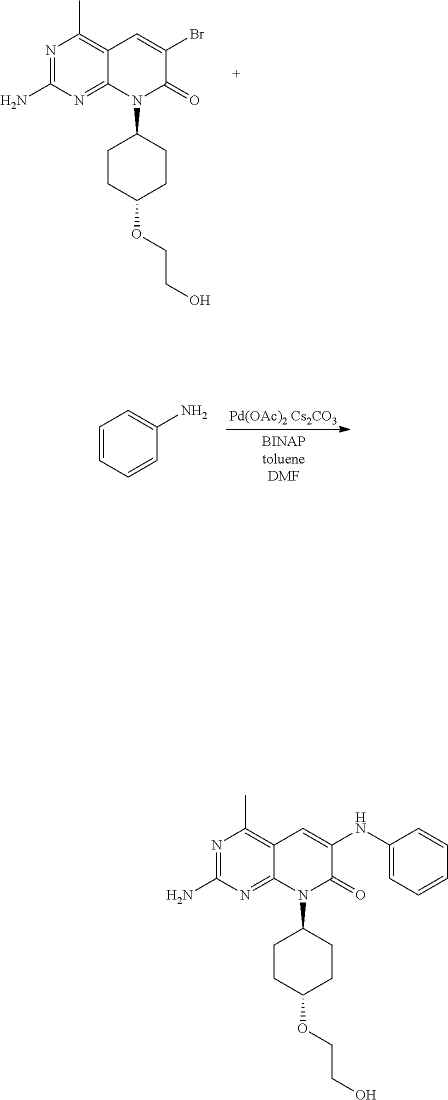 [SMILES: Cc1nc(N)nc2c1cc(Br)c(=O)n2[C@H]1CC[C@H](OCCO)CC1 ; Nc1ccccc1 ; "Pd(OAc)2 Cs2CO3" ; "BINAPtolueneDMF" ; Cc1nc(N)nc2c1cc(Nc1ccccc1)c(=O)n2[C@H]1CC[C@H](OCCO)CC1]

A flask containing a mixture of 2-amino-6-bromo-8-(trans-4-(2-hydroxyethoxy)cyclohexyl)-4-methylpyrido[2,3-d]pyrimidin-7(8H)-one (300 mg, 0.758 mmol), aniline (98.8 mg, 1.061 mmol), Cs₂CO₃ (345.7 mg, 1.061 mmol), Pd(OAc)₂ (8.5 mg, 0.0379 mmol), and BINAP (35.4 mg, 0.0557 mmol) was bubbled with argon, then DMF (3 ml) and toluene (3 ml) were added. This reaction mixture was stirred at 80° C. for 10 hours under the protection of argon. The reaction mixture was filtered through diatomite, the filtrate was diluted with EtOAc and water, the aqueous layer was extracted with EtOAc for three times. The combined organics were dried with MgSO₄, filtered and concentrated. The residue was purified by flash silica gel chromatography (0-5% methanol/DCM) to afford 2-amino-8-(trans-4-(2-hydroxyethoxy)cyclohexyl)-6-(phenylamino)-4-methylpyrido[2,3-d]pyrimidin-7(8H)-one.

¹HNMR (300 MHz, CDCl₃): 7.369 (t, 2H) 7.279 (s, 1H) 7.217 (d, 2H) 7.020-7.061 (m, 2H) 5.604 (s, 1H) 4.992 (s, 2H) 3,760 (t, 2H) 3.624-3.656 (m, 2H) 3.461-3.505 (m, 1H) 2.817 (s, 2H) 2.523 (s, 3H) 2.216 (d, 2H) 1.719 (t, 8H) 1.490 (d, 3H) 1.215-1.279 (m, 3H);

M+H: 410

Methods:

1. Cell-Based Assays:

Cells were seeded in 24-well plates with a concentration of 10×10⁵ cells/mL/well, and treated with the designated agents in the next day. After 72 hours treatment, cells were harvested and stained using the trypan blue exclusion method and measured using the TC-10™ automated cell counter (Bio-rad).

2. Western Blot Protocol

a. Harvest Cells and Run Cell Extracts on a SDS-PAGE Gel

Cells were harvested bytrypsinization and centrifugation at 500 rpm for 5 min and the supernatant was removed. Cell pellet was resuspended with appropriate volume of 1× lysis buffer (10× lysis buffer from Cell Signaling Technology, Catalog #9803, Boston, Mass.) containing protease inhibitors (Protease Inhibitor Cocktail Tablets, Roche, cat#11 697 498 001 and PMSF, NaF, Na3VO3, Leupeptin and pepstatin A), then directly sonicated the suspension for 5 seconds. Protein concentration was measured using Pierce BAC protein assay kit (catalog #23227). The protein sample was read with EL800 of Bio-TEK at 560 nM wave length and the concentration was calculated. 60 ug protein of each sample was loaded on an 8-15% SDS-PAGE and run at 120V for an appropriate period till the dye front reached the bottom of the gel.

b. Transfer the Protein from the Gel to a Membrane

Placed a cassette using pre-wet Watman paper(bio-rad, catalog #1703932), gel and membrane (PVDF membrane with pre-soak in methanol for 3 min, then equilibrated in transfer buffer for 5 min), and performed the transfer using bio-rad transfer system at 100v for 1.5 hr at cool room.

c. Block the Membrane

Removed the membrane from the transfer apparatus and placed it in the blocking buffer [5% non-fat dry milk in 1×TBS-T containing NaCl 8 g, Tris-Cl 2.37 g, TrisBase 0.6 g in 1 liter of water (pH 7.6) and 1 ml of Tween-20]. Incubated the membrane in the blocking buffer on a shaker for 0.5-1 hr at room temperature.

d. Primary Antibody

Diluted the primary antibody at 1:1000 using TBS-T containing 5% non-fat dry milk, and

incubated the membrane in the primary antibody solution on a shaker overnight at 4° C. Then removed the primary antibody solution, and washed the membrane with 1×TBS-T 3 times for 10 min each time.

e. Secondary Antibody, Substrate Addition and Film Explosure

Diluted the second antibody (cell signaling technology cat#7076 anti-mouse IgG, HRP-linked antibody, or cat#7074, anti-rabbit IgG, HRP-linked antibody) at 1:3000 using TBS-T containing 5% non-fat dry milk. Decanted the wash buffer and incubated the membrane in the prepared secondary antibody solution on shaker for 2 hr at room temperature. Decanted the secondary antibody solution and washed thoroughly with 1×TBS-T for 3 times for 10 min each time. Prepared for substrate solution: luminal/enhancer solution mix with stable peroxide solution (Amersham ECL Plus™ Western Blotting Detection Reagents. Cat#: RPN2132). Total amount of substrate solution needed was approximate 2 ml per membrane. Placed the membrane with the protein side up on a flat Saran wrap, and dropped the prepared substrate solution over the membrane, and incubated the membrane for 2 minutes. Then removed the substrate solution completely and placed the membrane between two pieces of transparencies and removed air bubbles. Exposed the membrane to X-ray film (Amersham Hyperfilm ECL, cat#28906838) in dark room, and developed the film in a developer.

### Results:

| | | LNCaP | T47D |
|---|---|---|---|
| | | (with PTEN-null, | (with PIK3CA |
| | | prostate tumor | mutation, breast |
| | | cell line), | tumor cell line), |
| | | IC50 (nM) | IC50 (nM) |
| | | (using cell | (using Westin blot |
| | | trypan blue | to detect the |
| | | exclusion assay | downstream |
| Example | | to detect the | targets of |
| No. | Compound Structure | cell viability) | PI3K/mTOR) |
| 1 | 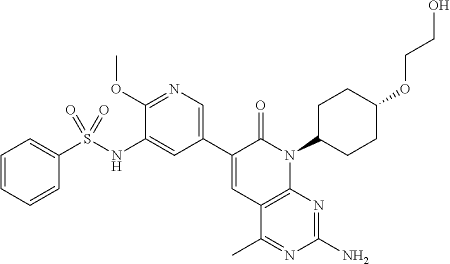 | 35 | 12 |
| 2 | 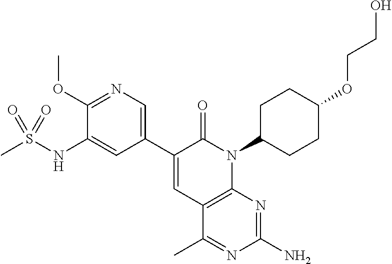 | 150 | |
| 3 | 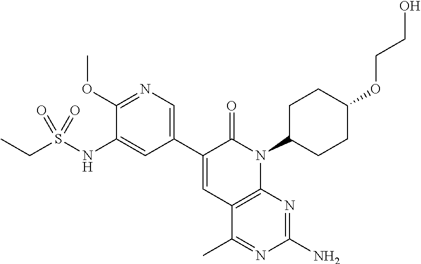 | 140 | |
| 4 | 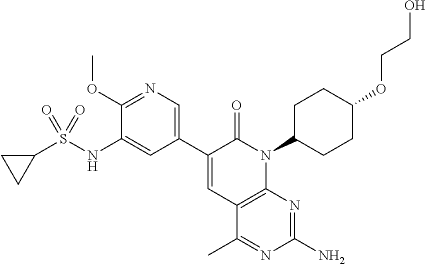 | 130 | |
| 5 | 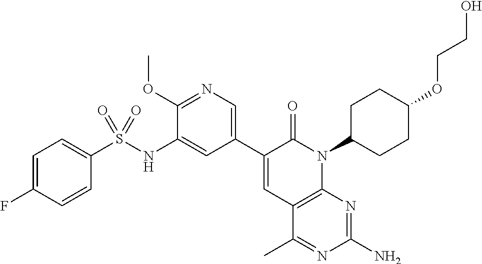 | 55 | |
| 6 | 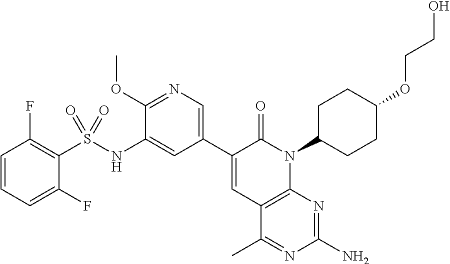 | 60 | |
| 7 | 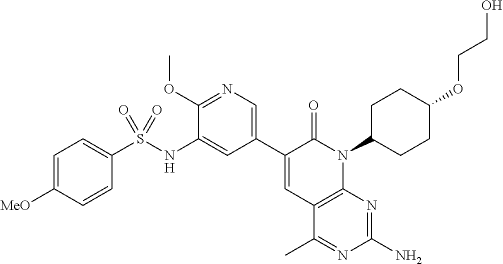 | 40 | 12 |
| 8 | 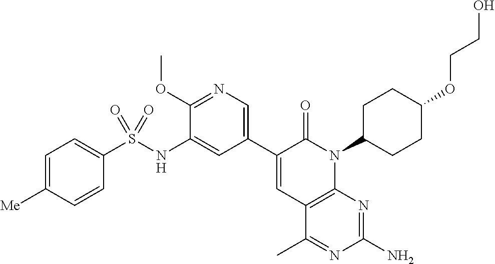 | 45 | 15 |
| 9 | 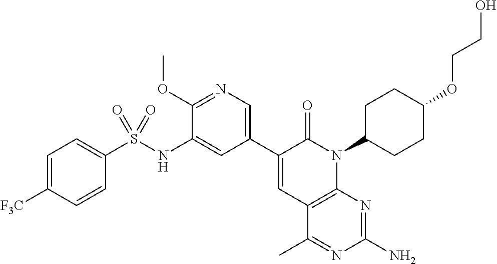 | | |
| 10 | 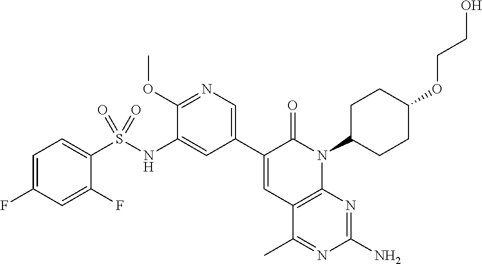 | | |
| 11 | 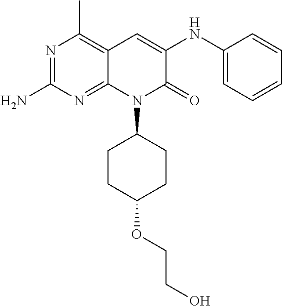 [SMILES: Cc1nc(N)nc2c1cc(Nc1ccccc1)c(=O)n2[C@H]1CC[C@H](OCCO)CC1] | | |
| PF-1502 | 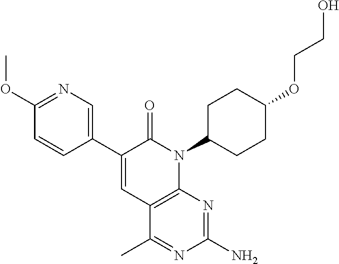 | 75 | 85 |

What is claimed is:

1. A compound of Formula I: 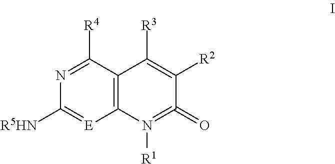
wherein:
R¹ is C₁-C₆ alkyl, C₃-C₇ cycloalkyl, C₂-C₆ heterocyclic alkyl, C₆-C₁₀ aryl, or C₃-C₁₀ heteroaryl, wherein said C₁-C₆ alkyl, C₃-C₇ cycloalkyl, C₂-C₆ heterocyclic alkyl, C₆-C₁₀ aryl, and C₃-C₁₀ heteroaryl are optionally and independently substituted with —OR, C₁-C₆ alkyl, C₃-C₇ cycloalkyl, C₂-C₆ alkynyl, C₆-C₁₀ aryl, C₂-C₆ heterocyclic alkyl, or C₃-C₁₀ heteroaryl, wherein said C₁-C₆ alkyl, C₃-C₇ cycloalkyl, C₂-C₆ alkynyl, C₆-C₁₀ aryl, C₂-C₆ heterocyclic alkyl, and C₃-C₁₀ heteroaryl are further optionally and independently substituted with 0-3 groups selected from halogen, —OR⁶, C₁-C₆ alkyl, C₃-C₇ cycloalkyl, —CN, —COOR⁷, —CONR⁸R⁹, —NR¹⁰C(O)R¹¹, —NR¹²S(O)ₓR¹³, —S(O)ₓNR¹⁴R¹⁵, and —NR¹⁶R¹⁷;
R is H, C₁-C₆ alkyl, C₂-C₆ heterocyclic alkyl, or C₃-C₇ cycloalkyl, wherein said C₁-C₆ alkyl, C₂-C₆ heterocyclic alkyl and C₃-C₇ cycloalkyl are optionally and independently substituted with 0-3 groups selected from halogen, C₂-C₆ heterocyclic alkyl, C₆-C₁₀ aryl, C₃-C₁₀ heteroaryl, —OR⁶, —NR¹⁶R¹⁷, C₁-C₆ alkyl, C₃-C₇ cycloalkyl, —CN, —COOR⁷, —CONR⁸R⁹, —NR¹⁰C(O)R¹¹, —NR¹²S(O)ₓR¹³, —S(O)ₓNR¹⁴R¹⁵, and —NR¹⁶R¹⁷;
R² is C₂-C₆ heterocyclic alkyl, C₆-C₁₀ aryl, —NH—C₆-C₁₀ aryl or C₃-C₁₀ heteroaryl, wherein said C₂-C₆ heterocyclic alkyl, C₆-C₁₀ aryl, —NH—C₆-C₁₀ aryl and C₃-C₁₀ heteroaryl are optionally and independently substituted with 0-3 groups selected from —OR⁶, halogen, —COOR⁷, —CONR⁸R⁹, —NR¹⁰C(O)R¹¹, —NR¹²S(O)ₓR¹³, —S(O)ₓNR¹⁴R¹⁵, —NR¹⁶R¹⁷, —CN, C₂-C₆ alkenyl, C₂-C₆ alkynyl, C₃-C₇ cycloalkyl, and C₁-C₆ alkyl;
R³ and R⁴ are independently H, C₁-C₆ alkyl, C₂-C₆ alkynyl, C₃-C₇ cycloalkyl, C₂-C₆ heterocyclic alkyl, C₆-C₁₀ aryl, halogen, or —CN;
R⁵ is C₁-C₆ alkyl, C₂-C₆ heterocyclic alkyl, or H;
E is CH or N;
each R⁶, R⁷, R⁸, R¹², R¹⁴, and R¹⁶ is independently H, C₁-C₆ alkyl, C₃-C₇ cycloalkyl, C₂-C₆ heterocyclic alkyl, C₆-C₁₀ aryl, or C₃-C₁₀ heteroaryl;
each R⁹, R¹⁰, R¹¹, R¹³, R¹⁵, and R¹⁷ is independently H, C₆-C₁₀ aryl, C₃-C₁₀ heteroaryl, C₂-C₆ heterocyclic alkyl, C₁-C₆ alkyl, or C₃-C₇ cycloalkyl, wherein said aryl, heteroaryl, and heterocyclic alkyl are optionally and independently substituted with 0-3 groups selected from halogen, —OR⁶, C₁-C₆ alkyl, C₃-C₇ cycloalkyl, —CN, and —COOR⁷;
each x is 0, 1 or 2;
wherein said heteroaryl is a 5- to 10-membered mono- or bicyclic aromatic ring containing 1-5 heteroatoms selected from O, S and N, said heterocyclic alkyl is a 3- to 10-membered mono- or bicyclic ring containing 1-5 heteroatoms selected from N, S and O, in which the point of attachment is carbon or nitrogen;
or a pharmaceutically acceptable salt, hydrate, or solvate thereof.

2. The compound of claim 1, wherein R³ is H, R⁴ is Me, R⁵ is H, and E is N.

3. The compound of claim 2, wherein the compound is represented by Formula III: 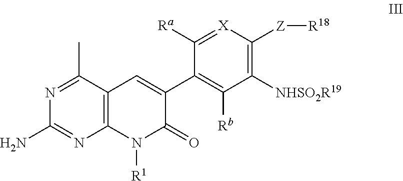
wherein:
Z is a bond, O, NH, NR, S, SO, or SO₂;
X is N or CH;
R¹⁸ is C₁-C₆ alkyl, C₃-C₇ cycloalkyl, C₂-C₆ heterocyclic alkyl, or H;
R¹⁹ is C₆-C₁₀ aryl, C₃-C₁₀ heteroaryl, C₁-C₆ alkyl, C₂-C₆ heterocyclic alkyl, or C₃-C₇ cycloalkyl, wherein said C₆-C₁₀ aryl, C₃-C₁₀ heteroaryl, C₁-C₆ alkyl, C₂-C₆ heterocyclic alkyl, and C₃-C₇ cycloalkyl are optionally and independently substituted with 0-3 groups selected from halogen, —OR, C₁-C₆ alkyl, C₂-C₆ heterocyclic alkyl, C₃-C₇ cycloalkyl, —CN, —COOR⁷, —CONR⁸R⁹, —NR¹⁰C(O)R¹¹, —NR¹²S(O)ₓR¹³, —S(O)ₓNR¹⁴R¹⁵, and —NR¹⁶R¹⁷;
R^{a} and R^{b} are independently C₁-C₆ alkyl, C₃-C₇ cycloalkyl, C₂-C₆ heterocyclic alkyl, H, halogen, or —CN.

4. The compound of claim 3, wherein the compound is represented by Formula IV:

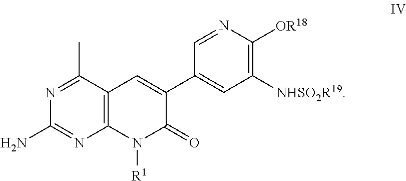

5. The compound of claim 4, wherein the compound is represented by Formula V: 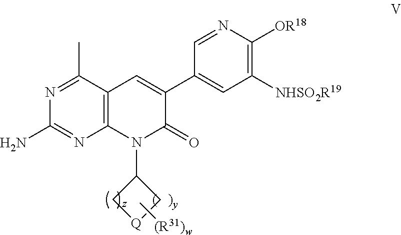
wherein:
Q is O or NR³²;
each R³¹ is independently C₁-C₆ alkyl, C₃-C₇ cycloalkyl, C₂-C₆ heterocyclic alkyl, C₆-C₁₀ aryl, C₃-C₁₀ heteroaryl, C₂-C₆ alkynyl, —CN, —OR, —COOR²⁰, —CONR²¹R²², —NR²³C(O)R²⁴, —NR²⁵S(O)ₘR²⁶, —S(O)ₘNR²⁷R²⁸, or —NR²⁹R³⁰, wherein said C₁-C₆ alkyl, C₃-C₇ cycloalkyl, C₂-C₆ heterocyclic alkyl, C₆-C₁₀ aryl, C₃-C₁₀ heteroaryl, and C₂-C₆ alkynyl are further optionally and independently substituted with up to 5 groups selected from halogen, C₁-C₆ alkyl, —CN, —OR, —COOR²⁰, —CONR²¹R²², —NR²³C(O)R²⁴, —NR²⁵S(O)ₘR²⁶, —S(O)ₘNR²⁷R²⁸, and —NR²⁹R³⁰;
y is 1, 2, or 3;
z is 1, 2, or 3;
w is 0, 1, or 2;
R³² is C₁-C₆ alkyl, C₃-C₇ cycloalkyl, C₂-C₆ heterocyclic alkyl, C₆-C₁₀ aryl, C₃-C₁₀ heteroaryl, —COOR²⁰, —CONR²¹R²², or —S(O)ₘNR²⁷R²⁸, wherein said C₁-C₆ alkyl, C₃-C₇ cycloalkyl, C₂-C₆ heterocyclic alkyl, C₆-C₁₀ aryl, and C₃-C₁₀ heteroaryl are further optionally and independently substituted with 1-5 groups selected from halogen, C₁-C₆ alkyl, —CN, —OR, —COOR²⁰, —CONR²¹R²², —NR²³C(O)R²⁴, —NR²⁵S(O)ₘR²⁶, —S(O)ₘNR²⁷R²⁸, and —NR²⁹R³⁰;
each m is independently 0, 1, or 2;
each R²⁰, R²¹, R²², R²³, R²⁴, R²⁵, R²⁶, R²⁷, R²⁸, R²⁹, and R³⁰ is independently H, C₆-C₁₀ aryl, C₃-C₁₀ heteroaryl, C₂-C₆ heterocyclic alkyl, C₁-C₆ alkyl, or C₃-C₇ cycloalkyl, wherein said aryl, heteroaryl, and heterocyclic alkyl are optionally and independently substituted with 0-3 groups selected from halogen, —OR⁶, C₁-C₆ alkyl, C₃-C₇ cycloalkyl, —CN, —COOR⁷, —CONR⁸R⁹, —NR¹⁰C(O)R¹¹, —NR¹²S(O)ₓR¹³, —S(O)ₓNR¹⁴R¹⁵, and —NR¹⁶R¹⁷.

6. The compound of claim 4, wherein the compound is represented by Formula VI: 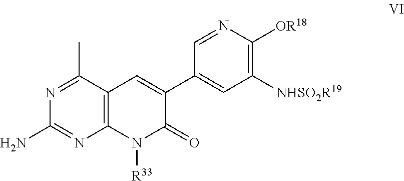
wherein:
R³³ is C₆-C₁₀ aryl or C₃-C₁₀ heteroaryl, wherein said aryl and heteroaryl are optionally and independently substituted with C₁-C₆ alkyl, C₃-C₇ cycloalkyl, C₂-C₆ heterocyclic alkyl, C₆-C₁₀ aryl, C₃-C₁₀ heteroaryl, C₂-C₆ alkynyl, —CN, —OR, —COOR²⁰, —CONR²¹R²², —NR²³C(O)R²⁴, —NR²⁵S(O)ₘR²⁶, —S(O)ₘNR²⁷R²⁸, or —NR²⁹R³⁰, wherein said C₁-C₆ alkyl, heterocyclic alkyl, C₃-C₇ cycloalkyl, C₂-C₆ alkynyl, and heterocyclyl are further optionally and independently substituted with up to 5 groups selected from halogen, C₁-C₆ alkyl, —CN, —OR, —COOR²⁰, —CONR²¹R²², —NR²³C(O)R²⁴, —NR²⁵S(O)ₘR²⁶, —S(O)ₘNR²⁷R²⁸, and —NR²⁹R³⁰;
each m is independently 0, 1, or 2; and
each R²⁰, R²¹, R²², R²³, R²⁴, R²⁵, R²⁶, R²⁷, R²⁸, R²⁹, and R³⁰ is independently H, C₆-C₁₀ aryl, C₃-C₁₀ heteroaryl, C₂-C₆ heterocyclic alkyl, C₁-C₆ alkyl, or C₃-C₇ cycloalkyl, wherein said aryl, heteroaryl, and heterocyclic alkyl are optionally and independently substituted with 0-3 groups selected from halogen, —OR⁶, C₁-C₆ alkyl, C₃-C₇ cycloalkyl, —CN, —COOR⁷, —CONR⁸R⁹, —NR¹⁰C(O)R¹¹, —NR¹²S(O)ₓR¹³, —S(O)ₓNR¹⁴R¹⁵, and —NR¹⁶R¹⁷.

7. The compound of claim 1, where the compound is
N-(5-(2-amino-8-(trans-4-(2-hydroxyethoxy)-cyclohexyl)-4-methyl-7-oxo-7,8-dihydropyrido [2,3-d]pyrimidin-6-yl)-2-methoxypyridin-3-yl)benzenesulfonamide;
N-(5-(2-amino-8-(trans-4-(2-hydroxyethoxy)cyclohexyl)-4-methyl-7-oxo-7,8-dihydropyrido [2,3-d]pyrimidin-6-yl)-2-methoxypyridin-3-yl)methanesulfonamide;
N-(5-(2-amino-8-(trans-4-(2-hydroxyethoxy)cyclohexyl)-4-methyl-7-oxo-7,8-dihydropyrido [2,3-d]pyrimidin-6-yl)-2-methoxypyridin-3-yl)ethanesulfonamide;
N-(5-(2-amino-8-(trans-4-(2-hydroxyethoxy)cyclohexyl)-4-methyl-7-oxo-7,8-dihydropyrido [2,3-d]pyrimidin-6-yl)-2-methoxypyridin-3-yl)cyclopropanesulfonamide;
N-(5-(2-amino-8-(trans-4-(2-hydroxyethoxy)cyclohexyl)-4-methyl-7-oxo-7,8-dihydropyrido [2,3-d]pyrimidin-6-yl)-2-methoxypyridin-3-yl)-4-fluorobenzenesulfonamide;
N-(5-(2-amino-8-(trans-4-(2-hydroxyethoxy)cyclohexyl)-4-methyl-7-oxo-7,8-dihydropyrido [2,3-d]pyrimidin-6-yl)-2-methoxypyridin-3-yl)-2,6-difluorobenzenesulfonamide;
N-(5-(2-amino-8-(trans-4-(2-hydroxyethoxy)cyclohexyl)-4-methyl-7-oxo-7,8-dihydropyrido [2,3-d]pyrimidin-6-yl)-2-methoxypyridin-3-yl)-4-methoxybenzenesulfonamide;
N-(5-(2-amino-8-(trans-4-(2-hydroxyethoxy)cyclohexyl)-4-methyl-7-oxo-7,8-dihydropyrido [2,3-d]pyrimidin-6-yl)-2-methoxypyridin-3-yl)-4-methylbenzenesulfonamide;
N-(5-(2-amino-8-(trans-4-(2-hydroxyethoxy)cyclohexyl)-4-methyl-7-oxo-7,8-dihydropyrido [2,3-d]pyrimidin-6-yl)-2-methoxypyridin-3-yl)-4-(trifluoromethyl)benzenesulfonamide;
N-(5-(2-amino-8-(trans-4-(2-hydroxyethoxy)cyclohexyl)-4-methyl-7-oxo-7,8-dihydropyrido [2,3-d]pyrimidin-6-yl)-2-methoxypyridin-3-yl)-2,4-difluorobenzenesulfonamide; or
2-amino-8-(trans-4-(2-hydroxyethoxy)cyclohexyl)-6-(phenylamino)-4-methylpyrido[2,3-d]pyrimidin-7(8H)-one.

8. A pharmaceutical composition comprising at least one compound of claim 1, or a salt, hydrate, isomer, or solvate thereof, and one or more pharmaceutically acceptable carriers and/or additives.

9. The pharmaceutical composition of claim 8 further comprising one or more cancer drugs in addition to the compound of claim 1.

10. A method for inhibiting protein kinase comprising administering a therapeutically effective amount of a compound of claim 1, or a salt, hydrate, isomer, or solvate thereof, or a pharmaceutical composition of claim 8.

11. The method of claim 10, wherein the protein kinase is PI3K or mTOR.

12. A method of treating abnormal cell growth in a human patient in need of, comprising administering a therapeutically effective amount of a compound of claim 1, or a salt, hydrate, isomer, or solvate thereof, or a pharmaceutical composition of claim 8.

13. The method of claim 12, wherein the abnormal cell growth is cancers.

14. A method of treating inflammation in a human patient in need of, comprising administering a therapeutically effective amount of a compound of claim 1, or a salt, hydrate, isomer, or solvate thereof, or a pharmaceutical composition of claim 8.
